(19) United States
(12) Patent Application Publication
(10) Pub. No.: US 2025/0295812 A1
(43) Pub. Date: Sep. 25, 2025

(54) TREATMENT FOR OSTEOARTHRITIS

(71) Applicant: Remedium Bio, Inc., Needham, MA (US)

(72) Inventors: Alexei Goraltchouk, Cambridge, MA (US); Alexey Seregin, Westford, MA (US)

(73) Assignee: Remedium Bio, Inc., Needham, MA (US)

(21) Appl. No.: 19/073,644

(22) Filed: Mar. 7, 2025

Related U.S. Application Data
(63) Continuation of application No. PCT/US2023/073641, filed on Sep. 7, 2023.
(60) Provisional application No. 63/404,294, filed on Sep. 7, 2022.

(51) Int. Cl. A61K 48/00; A61K 38/18; A61P 19/02

(52) U.S. Cl. CPC A61K 48/0058; A61K 38/1825; A61P 19/02

(57) ABSTRACT

The present invention applies at least in part to gene therapy treatments of diseases associated with cartilage loss, such as osteoarthritis. The treatments comprise administration of FGF-18 gene therapies, such as into the intra-articular space of joints to promote cartilage thickening.

### CROSS-REFERENCE TO RELATED APPLICATIONS

This application claims the benefit under 35 U.S.C. § 119(e) of U.S. Provisional Application No. 63/404,294, filed Sep. 7, 2022, entitled “TREATMENT FOR OSTEOARTHRITIS,” the disclosure of which is hereby incorporated by reference in its entirety.

### REFERENCE TO AN ELECTRONIC SEQUENCE LISTING

The contents of the electronic sequence listing (R087270002W000-SEQ-KVC.xml; Size: 29,526 bytes; and Date of Creation: Aug. 31, 2023) is herein incorporated by reference in its entirety.

### FIELD OF THE INVENTION

The present invention is related, at least in part, to gene therapy treatments of diseases associated with cartilage loss, such as osteoarthritis. The treatments comprise administration of FGF-18 gene therapies, such as into the intra-articular space of joints to promote cartilage thickening.

### SUMMARY OF THE INVENTION

Growth factors (e.g., Fibroblast Growth Factor 18 (FGF-18)) are proteins that regulate cell proliferation, migration, survival, differentiation, tissue deposition, turnover, and maintenance, among many other biological functions. In general, growth factor levels in tissues decline as a function of age. This decline is believed to be due at least in part to reduced gene expression mediated by one of many mechanisms of genetic silencing, general reduction in the cell density (which is hypothesized as a positive feedback loop with growth factor level decline), decreased efficiency and effectiveness in translation, and/or an increased proportion of senescent cells. Cellular density in tissues is correlated with tissue composition and physico-chemical properties. At least some of the decline in growth factor concentration has been associated with disease, tissue atrophy, and tissue degeneration. Osteoarthritis is predominantly a disease of aging and progressive cartilage loss leading to debilitating pain and loss of function. This process occurs in humans, non-human primates, as well as other animals including horses, dogs, cats, and pigs. While several genetic elements have been hypothesized as contributing factors, by far the strongest predictive factor of osteoarthritis prevalence and degree of progression is age.

Currently, there are no approved treatments capable of halting or reversing cartilage loss in osteoarthritis; however, recent clinical studies have demonstrated the ability of exogenous FGF-18 protein to promote and increase in cartilage thickness in a dose-dependent manner relative to placebo. For example, intra-articular FGF-18 protein injections have been demonstrated to reduce the rate of cartilage loss and in some treatment regimens increase cartilage thickness relative to placebo in controlled, randomized, clinical trials. While protein injection as a means of growth factor supplementation, substitution, or replacement has thus far demonstrated efficacy in reversing disease progression, the treatment paradigm remains challenging with up to 12 yearly injections required with a treatment regimen of 3-weekly injections up to every 6 months of bi-lateral treatment of knee osteoarthritis.

Provided herein are compositions and methods for treating cartilage disorders in a subject using gene therapy including a nucleic acid encoding a Fibroblast Growth Factor 18 (FGF-18) polypeptide. As used herein, a “cartilage disorder” is any condition where cartilage thickening, growth, regeneration and/or repair would be beneficial and/or one where there is cartilage loss, degeneration and/or damage. Cartilage disorders include, but are not limited to, degenerative diseases of cartilage and the meniscus, meniscal tears, focal cartilage lesions, and osteoarthritis (e.g., secondary osteoarthritis). The subject may be a mammal. The subject may be a human, dog, cat, cow, sheep, goat, horse, or other animals. The nucleic acid may be under the regulation of an optimal promoter and/or regulatory sequences. The nucleic acid may also encode a secretion signal. In some embodiments, the gene therapy is administered by local or intra-articular administration. In some embodiments, the gene therapy is in an optimal dose range.

It has also been recognized that it can be beneficial to dose a gene therapy locally and in a manner where the dose is correlated to the amount of target tissue, such as at the time of administration. The surface areas of joints have been generally characterized in a broad range of animals. As an example, the total articular surface of the knee joint cartilage plates has been demonstrated to range between 102 and 163 cm² for human adults with a mean of 121 and a standard deviation of 14.1 cm². The patella has been estimated to average an articular surface of 12 cm², and the total volume of cartilage in the human knee has been estimated to average at approximately 23.3 cm³ for human adults. Dhollander et al. have demonstrated by 3-dimensional MRI analysis that the mean knee cartilage volume of male and female Beagle dogs weighing between 7.2 and 17.1 kg ranged from 319.7 to 647.3 mm³. Similarly, rabbit, sheep, dog, goat, and horse cartilage thickness has been estimated to be 0.3 mm, 0.4-0.5 mm, 0.6-1.3 mm, 0.7-1.5 mm, and 1.5-2.0 mm, respectively. As such, knowing the volume, thickness, or surface area of the cartilage and adjusting the gene therapy dose appropriately can provide optimal dosing for a regenerative gene therapy intended to halt or reverse cartilage loss, such as in osteoarthritis. Any one of the compositions and methods provided herein can be or result in the administration of such an optimal dose. Also provided herein are methods for determining such an optimal dose as well as compositions whereby a gene therapy is in such an optimal dose amount.

Furthermore, clinical data demonstrates that if treatment is discontinued cartilage gains are reversed and cartilage loss resumes. Thus, optimal and/or more durable treatment approaches are needed to mitigate progressive cartilage loss in osteoarthritis and prevent osteoarthritis-related comorbidities. The compositions and methods provided herein can be such optimal and/or durable treatments.

Aspects of the present disclosure relate to a genetic construct comprising, a nucleic acid encoding a FGF-18 polypeptide, a promoter and, optionally, a regulatory element (such as a post-translational regulatory element) (e.g., is from the WPRE sequence family, encodes a PolyA signal, is an enhancer, is a translation termination sequence, is a sequence that promotes binding of one or more DNA binding proteins).

In some embodiments of any one of the compositions or methods provided herein, the FGF-18 polypeptide is mammalian, such as human or non-human primate. In some embodiments of any one of the compositions or methods provided herein, the FGF-18 polypeptide is a human, dog, cat, cow, sheep, goat or horse FGF-18 polypeptide. In some embodiments of any one of the compositions or methods provided herein, the FGF-18 polypeptide is encoded by the sequence of AF075292, AB007422, AF211188, BT019570, BTo19571, CH471062, BC006245, AY358811, NM_003862.2 or a portion thereof. In some embodiments of any one of the compositions or methods provided herein, the FGF-18 polypeptide is encoded by at least a part of the sequence of gene ID 8817 from the HGNC:3674.

In some embodiments of any one of the compositions or methods provided herein, the nucleic acid encodes another protein or portion thereof. In some embodiments of any one of the compositions or methods provided herein, the nucleic acid further encodes at least one auxiliary and/or regulatory sequence that facilitates expression. In some embodiments of any one of the compositions or methods provided herein, the nucleic acid further encodes at least one intron from another genetic sequence (e.g., human).

In some embodiments of any one of the compositions or methods provided herein, the promoter is a/an CMV promoter, CMV promoter with an MVM1 intron, CAG promoter, EF1 alpha promoter, UBC promoter, CBh promoter, MSCV promoter, hPGK promoter, SFFV promoter, or SV40 promoter. In some embodiments of any one of the compositions or methods provided herein, the promoter is a constitutive promoter (e.g., mammalian). In some embodiments of any one of the compositions or methods provided herein, the promoter drives expression in at least one cell type at least transiently present within a joint or tissues surrounding a joint. In some embodiments of any one of the compositions or methods provided herein, the promoter is an inducible promoter. In some embodiments of any one of the compositions or methods provided herein, the inducible promoter up- or down-regulates expression in response to external or internal stimuli (e.g., inflammation, heat, light, stress, administration of steroids, tetracycline, antibiotics, rapamycin, ganciclovir, acyclovir) or is inducible by up- or down-regulated heat, ROS, NOS or cytokine release. In some embodiments of any one of the compositions or methods provided herein, the promoter is a circadian rhythm or cycling promoter, such as in response to cortisol levels, the menstrual cycle, the diumal cycle, with the level of exercise, etc. (e.g., that changes its level of activity by at least 2%, 5% or 10% with some periodicity, such as from hours to months, including weekly). In some embodiments of any one of the compositions or methods provided herein, the promoter is a tissue-specific promoter. In some embodiments of any one of the compositions or methods provided herein, the promoter is a chondrocyte-specific promoter.

In some embodiments of any one of the compositions or methods provided herein, the promoter is a synoviocyte-specific promoter.

In some embodiments of any one of the compositions or methods provided herein, the regulatory sequence element is one or more of Argc1, Col2a1, Col6a1, Col10a1, Col11a2, Matn1, Gdf5, IL1B, and Prx1. In some embodiments of any one of the compositions or methods provided herein, the regulatory element is one or more of Adam12, Alpha-SMA, Col1a1, Col1a2, FGF18, FGF10, FGF-2, FoxD1, Fsp1, FoxJ1, Gli1, PDGFa, PDGFb, PDFR-alpha, PDGFR-beta, Twist2, and TCF4. In some embodiments of any one of the compositions or methods provided herein, the regulatory element is an intron, part of an intron, post-translational regulatory element, enhancer, repressor, a genetic sequence capable of forming multi-dimensional structures with parts of the genome or the genetic construct itself, or generally a genetic regulatory element.

Aspects of the present disclosure relate to a composition comprising any genetic construct described herein, wherein the nucleic acid is comprised in a delivery vector.

In some embodiments of any one of the compositions or methods provided herein, the delivery vector is a polyelectrolytic complex or polypeptide, viral (e.g., an adeno-associated virus (e.g., AAV2), an adenovirus, lentivirus, herpes simplex virus, pox virus, measles virus, alphavirus, or mimivirus), polymeric or lipid carrier, optionally, coupled to a ligand (such as to enhance selectivity and/or specificity and/or to target to a tissue or cell type) (e.g., at least a part of an Fc fragment, at least a part of a cytokine, at least a part of a growth factor, at least a part of a growth factor receptor, at least a part of a molecule that increases the residence time of a growth factor, or at least a part of a molecule that increases binding affinity to a receptor).

In some embodiments of any one of the compositions or methods provided herein, the viral carrier is from a virus with a synthetic or hybrid capsid. In some embodiments of any one of the compositions or methods provided herein, the viral carrier is from a virus with a natural or synthetic capsid, optionally, conjugated to a ligand. In some embodiments of any one of the compositions or methods provided herein, the viral carrier is comprised of more than one virus type. In some embodiments of any one of the compositions or methods provided herein, at least 5% of capsids of the viral carrier are full capsids.

In some embodiments of any one of the compositions or methods provided herein, the delivery vector is polymeric, optionally, conjugated to a ligand. In some embodiments of any one of the compositions or methods provided herein, the delivery vector is a polyelectrolytic complex comprising at least one polymer, optionally, conjugated to a ligand.

In some embodiments of any one of the compositions or methods provided herein, the polymers are cationic polymers, anionic polymers and/or non-ionic polymers.

In some embodiments of any one of the compositions or methods provided herein, the delivery vector comprises chitosan, polyethyleneimine, or a polypeptide with an overall positive charge. In some embodiments of any one of the compositions or methods provided herein, the ligand targets any one of the tissues or cell types described herein. In some embodiments of any one of the compositions or methods provided herein, the delivery vector is a lipid nanoparticle or liposome, optionally, conjugated to a ligand. In some embodiments of any one of the compositions or methods provided herein, the lipid carrier comprises up to 60% by molar ratio cholesterol. In some embodiments of any one of the compositions or methods provided herein, the lipid carrier comprises up to 80% by molar ratio a cationic or ionizable lipid. In some embodiments of any one of the compositions or methods provided herein, wherein the lipid carrier comprises glycerides, polyglyceryls and/or polyoxylglycerides. In some embodiments of any one of the compositions or methods provided herein, the lipid carrier comprises an oil/water nanoemulsion or an oil/water microemulsion. In some embodiments of any one of the compositions or methods provided herein, the lipid carrier is a nanocapsule, a self-nanoemulsifying or self-microemulsifying system, a micelle, a lipid-polymer hybrid or comprises a biopolymer or a biomimetic.

In some embodiments of any one of the compositions or methods provided herein, the ligand comprises peptides, proteins, polysaccharides, small molecules, or combinations thereof (e.g., for targeting, increased uptake, or increased in vivo residence time).

Aspects of the present disclosure relate to a method of administering any one of the genetic constructs or compositions described herein, to a subject in need thereof. In some embodiments of any one of the methods provided herein, the subject has or is at risk of a cartilage disorder, cartilage loss and/or is in need of cartilage regeneration. In some embodiments of any one of the methods provided herein, the subject has or is at risk of osteoarthritis. In some embodiments of any one of the methods provided herein, the subject has a meniscal tear.

In some embodiments of any one of the methods provided herein, the genetic construct or composition is administered locally or intra-articularly. In some embodiments of any one of the methods provided herein, the genetic construct or composition is administered to the meniscus of a joint.

In some embodiments of any one of the methods provided herein, the subject is a human subject. In some embodiments of any one of the methods provided herein, the subject is a horse, dog or cat.

In some embodiments of any one of the compositions or methods provided herein, the dose of the genetic construct is at a dose that is or was calculated or adjusted using an average joint size (e.g., average joint surface area or average joint volume), average articular cartilage volume, weight, age and/or disease state of or representative of the subject relative to another subject, such as of a different species. In another aspect, a composition comprising one or more of any one of the doses provided herein (or can provide one or more of any one of the doses provided herein) is provided.

In some embodiments of any one of the methods provided herein, the metric representative of the subject is determined for the subject, optionally, the method further comprises determining the average joint size (e.g., average joint surface area or average joint volume), average articular cartilage volume, weight, age and/or disease state for the subject. In some embodiments of any one of the methods provided herein, the metric representative of the subject is of another subject representative of the subject, such as a healthy subject or other subject of the same species, optionally, the method further comprises determining the average joint size (e.g., average joint surface area or average joint volume), average articular cartilage volume, weight, age and/or disease state for the other subject.

In some embodiments of any one of the compositions or methods provided herein, the dose is or was calculated or adjusted using an average joint size (e.g., average joint surface area or average joint volume) or average articular cartilage volume in relation to weight. In some embodiments of any one of the compositions or methods provided herein, the dose is or was calculated or adjusted using an average joint size (e.g., average joint surface area or average joint volume) or average articular cartilage volume in relation to age. In some embodiments of any one of the compositions or methods provided herein, the dose is or was calculated or adjusted using an average joint size (e.g., average joint surface area or average joint volume) or average articular cartilage volume in relation to weight and age. In some embodiments of any one of the compositions or methods provided herein, the dose is or was calculated or adjusted using an average joint size (e.g., average joint surface area or average joint volume) or average articular cartilage volume in relation to disease state. In some embodiments of any one of the compositions or methods provided herein, the dose is or was calculated or adjusted using an average joint size (e.g., average joint surface area or average joint volume) or average articular cartilage volume in relation to disease state and weight. In some embodiments of any one of the compositions or methods provided herein, the dose is or was calculated or adjusted using an average joint size (e.g., average joint surface area or average joint volume) or average articular cartilage volume in relation to disease state and age. In some embodiments of any one of the compositions or methods provided herein, the dose of the genetic construct is at a dose that is or was calculated or adjusted using an average expression ratio (e.g., based on the promoters and/or regulatory elements of the construct) (such as an average promoter ratio and/or regulatory element ratio).

In some embodiments of any one of the compositions or methods provided herein, the dose of the genetic construct is between 2×10⁹ to 1×10¹¹ genome copies/joint (rat) or equivalent as provided herein, such as a human, horse, dog or cat equivalent. In some embodiments of any one of the compositions or methods provided herein, the dose of the genetic construct is between 1×10¹⁰ to 6×10¹¹ genome copies/joint (human) or equivalent as provided herein, such as a horse, dog or cat equivalent. In some embodiments of any one of the compositions or methods provided herein, the dose of the genetic construct is between 5×10¹¹ to 3×10¹³ genome copies/joint (human) or equivalent as provided herein, such as a horse, dog or cat equivalent. In some embodiments of any one of the compositions or methods provided herein, the dose of the genetic construct is between 5×10¹¹ to 3.5×10¹² genome copies/joint (dog) or equivalent as provided herein, such as a human, horse or cat equivalent. In some embodiments of any one of the compositions or methods provided herein, the dose of the genetic construct is between 1×10¹⁰ to 7×10¹⁰ genome copies/joint (dog) or equivalent as provided herein, such as a human, horse or cat equivalent. In some embodiments of any one of the compositions or methods provided herein, the dose of the genetic construct is between 1×10¹² to 8.5×10¹³ genome copies/joint (horse) or equivalent as provided herein, such as a human, dog or cat equivalent. In some embodiments of any one of the compositions or methods provided herein, the dose of the genetic construct is between 3×10¹⁰ to 2×10¹² genome copies/joint (horse) or equivalent as provided herein, such as a human, dog or cat equivalent. In some embodiments of any one of the compositions or methods provided herein, the dose of the genetic construct is between 1×10⁸ to 6.5×10⁹ genome copies/joint/kg (human) or equivalent as provided herein, such as a horse, dog or cat equivalent. In some embodiments of any one of the compositions or methods provided herein, the dose of the genetic construct is between 5×10⁹ to 3.5×10¹¹ genome copies/joint/kg (human) or equivalent as provided herein, such as a horse, dog or cat equivalent. In some embodiments of any one of the compositions or methods provided herein, the dose of the genetic construct is between 2×10⁴ genome copies/joint/kg to 6.3×10¹³ genome copies/kg, or equivalent as provided herein. In some embodiments of any one of the compositions or methods provided herein, the dose of the genetic construct is between 3.8×10⁴ genome copies/joint/kg to 4.7×10¹³ genome copies/kg (humans), or equivalent as provided herein. In some embodiments of any one of the compositions or methods provided herein, the dose of the genetic construct is between 4.5×10⁵ genome copies/joint/kg to 6.3×10¹³ genome copies/kg (horse), or equivalent as provided herein. In some embodiments of any one of the compositions or methods provided herein, the dose of the genetic construct is between 3.8×10⁴ genome copies/joint/kg to 1.2×10¹³ genome copies/kg (dog), or equivalent as provided herein. In some embodiments of any one of the compositions or methods provided herein, the dose of the genetic construct is between 2×10⁴ genome copies/joint/kg to 4.3×10¹² genome copies/kg (cat), or equivalent as provided herein. In some embodiments of any one of the compositions or methods provided herein, the dose of the genetic construct is between 5×10⁷ genome copies/knee, hip, or shoulder joint to 2×10¹⁴ genome copies/knee, hip, or shoulder joint (human), or equivalent as provided herein. In some embodiments of any one of the compositions or methods provided herein, the dose of the genetic construct is between 5×10⁷ genome copies/knee, hip, or shoulder joint to 5×10¹³ genome copies/knee, hip, or shoulder joint (horse), or equivalent as provided herein. In some embodiments of any one of the compositions or methods provided herein, the dose of the genetic construct is between 1×10⁵ genome copies/knee, hip, or shoulder joint to 1×10¹² genome copies/knee, hip, or shoulder joint (dog), or equivalent as provided herein. In some embodiments of any one of the compositions or methods provided herein, the dose of the genetic construct is between 5×10⁴ genome copies/knee, hip, or shoulder joint to 7×10¹¹ genome copies/knee, hip, or shoulder joint (cat), or equivalent as provided herein. In some embodiments of any one of the compositions or methods provided herein, the dose of the genetic construct is between 4×10⁷ genome copies/knee, hip, or shoulder joint to 2×10¹⁴ genome copies/knee, hip, or shoulder joint, or equivalent as provided herein. In some embodiments of any one of the compositions or methods provided herein, the dose of the genetic construct is between 5×10⁸ genome copies/knee, hip, or shoulder joint to 1×10¹³ genome copies/knee, hip, or shoulder joint, or equivalent as provided herein. In some embodiments of any one of the compositions or methods provided herein, the dose of the genetic construct is between 5×10⁸ genome copies/knee, hip, or shoulder joint to 8×10¹² genome copies/knee, hip, or shoulder joint, or equivalent as provided herein.

Aspects of the present disclosure relate to a method comprising, determining or adjusting a dose based on using an average joint size (e.g., average joint surface area or average joint volume), average articular cartilage volume, weight, age and/or disease state of or representative of the subject relative to another subject, such as of a different species.

In some embodiments of any one of the methods provided herein, the metric representative of the subject is determined for the subject, optionally, the method further comprises determining the average joint size (e.g., average joint surface area or average joint volume), average articular cartilage volume, weight, age and/or disease state for the subject. In some embodiments of any one of the methods provided herein, the metric representative of the subject is of another subject representative of the subject, such as a healthy subject or other subject of the same species, optionally, the method further comprises determining the average joint size (e.g., average joint surface area or average joint volume), average articular cartilage volume, weight, age and/or disease state for the other subject.

In some embodiments of any one of the compositions or methods provided herein, the dose is or was calculated or adjusted using an average joint size (e.g., average joint surface area or average joint volume) or average articular cartilage volume in relation to weight. In some embodiments of any one of the compositions or methods provided herein, the dose is or was calculated or adjusted using an average joint size (e.g., average joint surface area or average joint volume) or average articular cartilage volume in relation to age. In some embodiments of any one of the compositions or methods provided herein, the dose is or was calculated or adjusted using an average joint size (e.g., average joint surface area or average joint volume) or average articular cartilage volume in relation to weight and age. In some embodiments of any one of the compositions or methods provided herein, the dose is or was calculated or adjusted using an average joint size (e.g., average joint surface area or average joint volume) or average articular cartilage volume in relation to disease state. In some embodiments of any one of the compositions or methods provided herein, the dose is or was calculated or adjusted using an average joint size (e.g., average joint surface area or average joint volume) or average articular cartilage volume in relation to disease state and weight. In some embodiments of any one of the compositions or methods provided herein, the dose is or was calculated or adjusted using an average joint size (e.g., average joint surface area or average joint volume) or average articular cartilage volume in relation to disease state and age. In some embodiments of any one of the compositions or methods provided herein, the dose is or was calculated or adjusted using an average expression ratio (e.g, based on the promoters and/or regulatory elements of the construct) (such as an average promoter ratio and/or regulatory element ratio).

Aspects of the present disclosure relate to a composition comprising any of the genetic constructs provided herein at any one of the doses provided herein and a pharmaceutically acceptable carrier.

Aspects of the present disclosure relate to a genetic construct as described in any one of the Examples described herein.

### BRIEF DESCRIPTION OF THE DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1 shows cytocompatibility and transfection efficiency of AAV2 vectors with primary human chondrocytes and synoviocytes (Cytocompatibility: pTime>0.05, pDose>0.05, Transfection Efficiency: pTime<0.05, pDose<0.05, ANOVA).

FIG. 2 shows proliferation of human chondrocytes upon stimulation by rhFGF18 protein (Left) and AAV2-FGF18 (Right) (Protein: pDose<0.05, PType>0.05, ANOVA; AAV: PDose<0.05, ANOVA).

FIG. 3 shows the effect of AAV2-FGF18 gene therapy and rhFGF18 protein on chondrocytes in transwell culture with synoviocytes relative to AAV2-GFP negative control (ANOVA, p<0.05; Tukey's Pairwise Comparison grouping indicated by letter code, pcrit=0.05).

FIG. 4 shows a gene set up- and down-regulated by AAV2-FGF18 treated chondrocytes in comparison to the full set of genes up- and down-regulated by rhFGF18 protein treated chondrocytes. *ESM1 was also upregulated by AAV2-GFP control over PBS, however, by less than 3×.

FIG. 5 shows the effect of AAV2-FGF18 gene therapy (Left) and rhFGF18 protein treatment (Right) on gene expression in primary human chondrocytes relative to PBS treatment (* indicates statistical significance using RESeq2, pcrit=0.01).

FIG. 6 shows, from left to right: 1) AAV2-nLuc bioluminescent reporter analysis, 4 months following initial dosing, 2) AAV2-FGF18 Active Group hFGF18 antibody immunohistochemical staining, 3) AAV2-GFP Control hFGF18 antibody immunohistochemical staining, 4) AAV2-GFP Control hFGF18 antibody immunohistochemical staining without a primary antibody (Ab) serving as a negative control.

FIG. 7 shows safranin-O-stained sagittal histology sections of rat knee joints injected with AAV2-GFP (Top), AAV2-FGF18 (Middle), and rhFGF18 Protein (Bottom) showing anatomical locations of cartilage thickness measurement (translucent, white boxes). Locations for Tibia were selected as 6 uniformly spaced, equal width rectangles, while the location for the meniscal tip was taken as a single width at the thickest point of the tip.

FIG. 8 shows weight-normalized cartilage thickness average by anatomical location following administration of rhFGF18 protein positive control, three doses of AAV2-FGF18 (Dose 1=2×10⁹ vg/joint, Dose 2=1×10¹⁰ vg/joint, Dose 3=1×10¹¹ vg/joint), and AAV2-GFP negative control. All groups within anatomical location reach statistical significance against the AAV2-GFP negative control (ANOVA, p<0.05; Tukey's pairwise comparison grouping indicated by letter code, pcrit=0.05).

FIG. 9 shows joint diameters at the 1 month and 2 months study timepoint relative to the no-injection baseline; AAV2-FGF18 single injection, AAV2-GFP control single injection, rhFGF18 protein bi-weekly injections (ANOVA, p<0.05; Tukey's pairwise comparison grouping indicated by letter code, pcrit=0.05, dashed red lines indicate 99% confidence interval of the mean of unoperated control).

FIG. 10 shows gene expression changes following administration of rhFGF18 (Left) and AAV2-FGF18 (Right) relative to PBS negative control.

FIG. 11 shows hyaline cartilage and Fibrocartilage associated pathways affected by AAV2-FGF18 administration, as determined by RNA-Seq analysis. Direct hyaline cartilage promoting effects observed in RNA-Seq analysis: Lubricin (PRG4) and Collagen 2 (COL2A1). Indirect hyaline cartilage promoting effects: ADAMTS1, 5, 15, and Matrix Metalloprotease 2 (MMP2). Direct fibrocartilage suppressing effects: Collage 1 (COL1A1) and Lysine 6-oxidase (LOX). Indirect fibrocartilage suppressing effects: PTX3 and IGF1.

FIG. 12 shows weight normalized cartilage thickness by anatomic location.

### DETAILED DESCRIPTION OF THE INVENTION

Provided herein are compositions and methods for preventing, reducing, or reversing cartilage loss, such as in osteoarthritic joints. More specifically, provided herein are compositions and methods for preventing, reducing, or reversing cartilage loss, such as in osteoarthritic joints, by administering nucleic acid acids that encode a human growth factor, such as human FGF (e.g., FGF-18), or a functional portion thereof.

In one embodiment, a composition provided herein is administered via an intra-articular injection and delivers a nucleic acid encoding a FGF-18 polypeptide. As used herein, “FGF-18 polypeptide” includes any FGF-18 polypeptide that exhibits one or more functions as a full-length FGF-18 protein from any species, such as from humans, dogs, cats, cows, sheep, goat, horse, or other animals. FGF-18 polypeptides also include full-length FGF-18 proteins from any species as well as functional portions or fragments of such full-length FGF-18 proteins. FGF-18 polypeptides, thus, also include non-human primate FGF-18 proteins, full-length, or functional fragments or portions thereof. FGF-18 polypeptides also include mammalian or non-mammalian homologs, paralogs, or orthologs, mammalian or non-mammalian functional analogs, etc.

Accordingly, a “FGF-18 gene”, as used herein, refers to the sequence that encodes the FGF-18 polypeptide. Thus, a FGF-18 gene can encode a full-length FGF-18 protein from any species or a functional portion thereof. Examples of FGF-18 polypeptides, including functional portions, include, but are not limited to, AEENVDFRIH VENQTRARDD VSRKQLRLYQ LYSRTSGKHI QVLGRRISAR GEDGDKYAQL LVETDTFGSQ VRIKGKETEF YLCMNRKGKL VGKPDGTSKE CVFIEKVLEN NYTALMSAKY SGWYVGFTKK GRPRKGPKTR ENQQDVHFMK RYPKGQPELQ KPFKYTTVTK RSR (SEQ ID NO: 7), amino acids 28-207 of uniprot.org/uniprotkb/076093/entry#sequences, and Sprifermin (amino acids 28-196).

The sequence encoding a FGF-18 polypeptide is preferably under the regulation of a promoter, such as a constitutive, inducible, tissue-specific, or cycling promoter, and, optionally, is also under the regulation of a regulatory sequence.

In one embodiment, the nucleic acid is at least partially encased in a viral capsid with or without secondary modifications, a lipid-based carrier, or a polymer-based carrier capable of delivering a nucleic acid to the inside of nucleated cells. The sequences provided herein may be RNA, DNA, or a hybrid of RNA and DNA, or a chemically modified sequence based on RNA, DNA, or RNA and DNA.

In one embodiment, the FGF-18 gene and/or any other coding or non-coding sequences delivered in cis or in trans (e.g., other auxiliary non-coding RNA sequence or other coding sequences) may be under the regulation of a constitutive promoter. As used herein, a “constitutive promoter” drives expression without significant fluctuation in expression in a manner that is not tissue-, cell type-, or cell stage-dependent. In one embodiment, the constitutive promoter may be a CMV promoter with or without hybrid elements, such as, for example, the MVM1 intron, a CAG promoter, EF1-alpha promoter, UBC promoter, CBh promoter, MSCV promoter, hPGK, promoter, SFFV promoter, SV40 promoter, or generally a constitutive promoter that is capable of driving expression in at least one cell type at least transiently present within a joint or surrounding tissues including, as an example, the joint capsule, or a combination of one or more of the aforementioned promoters or functional elements thereof.

In another embodiment, the FGF-18 gene and/or any other coding or non-coding sequences delivered in cis or in trans (e.g., other auxiliary non-coding RNA sequence or other coding sequences) may be driven by an inducible promoter. Some examples of inducible promoters include LexA, AlcaA, araBAD, PtxA, SPLs, GAL7, TRE, or more generally, a steroid inducible promoter, a tetracycline inducible promoter, a rapamycin inducible promoter, a ganciclovir inducible promoter, an acyclovir inducible promoter, a temperature inducible promoter, a stress inducible promoter, a promoter inducible by increased oxidative state, a promoter induced by upregulation of one or more of ROS, NOS, cytokine, or another exogenously administered molecule or a combination or one or more functional elements of the aforementioned inducible promoters.

In another embodiment, it may be beneficial to drive expression at alternating levels with a periodicity and without the use of external stimuli. For example, the FGF-18 gene and/or any other coding or non-coding sequences delivered in cis or in trans (e.g., other auxiliary non-coding RNA sequence or other coding sequences) may be driven via the use of a cycling promoter. Examples of cycling promoters include promoters that alter their expression modulation levels as a function of some natural or semi-natural cycle of the organism or its surroundings such as, for example, the circadian cycle, a cycle with a weekly periodicity, monthly periodicity, the menstrual cycle, cortisol synthesis cycle, diurnal cycle, rest-activity cycle, or the level of exercise. In general, it may be beneficial that such a cycling promoter varies its activity by a minimum of 2%, a minimum of greater than 5%, or in some cases, such as the circadian cycle, cortisol response, diurnal cycle, menstrual cycle, or exercise, by at least 10% of an average or other activity (such as compared to a minimum or maximum or opposite periodic activity) level. Some specific examples of cycling promoters include without limitations one or more of or elements of the CLOCK promoter, BMAL 1 promoter, PER promoter, Cry promoter, NFIL3 promoter, DEC promoter, or the PPAR-gamma promoter.

In yet another embodiment, the FGF-18 gene and/or any other coding or non-coding sequences delivered in cis or in trans (e.g., other auxiliary non-coding RNA sequence or other coding sequences), can be driven by tissue-specific promoters that demonstrate at least preferential expression patterns within cells that are at least transient residents of the joint, joint capsule, or surrounding tissues. Examples of such promoters include promoters that display preferentially increased expression in chondrocytes, chondroblasts, synoviocytes, synovioblasts, fibroblasts, fibroblast-like synoviocytes, or cells of the chondrocyte or synoviocyte lineage. In one embodiment, the promoter may promote gene expression in connective tissue cells or resident immune cells of the joint, Joint capsule, or surrounding tissues. Some specific examples of tissue-specific promoters include one or more of or a functional fragment of a promoter or enhancer of Argc1, Col2a1, Col6a1, Col10a1, Col12, Matn1, Gdf5, IL1B, Prx1, Adam12, Alpha-SMA, Col1a1, Col1a2, FGF18, FGF10, FGF-2, FoxD1, Fsp1, FoxJ1, Gli1, PDGFa, PDGFb, PDFR-alpha, PDGFR-beta, Twist2, or TCF4.

In one embodiment, the FGF-18 gene encodes the full coding sequence of FGF-18 protein, or the cDNA sequence of the FGF-18 protein, or a functional portion thereof. The FGF-18 protein may be human FGF-18 protein. In one embodiment, at least a part of the following genetic sequences or a combination thereof, are encoded: AF075292, AB007422, AF211188, BT019570, BTo19571, CH471062, BC006245, AY358811, or NM_003862.2. The FGF-18 gene may be a codon optimized version of any one of the sequences provided herein. As an example, the FGF-18 polypeptide may be encoded by at least a part of the gene ID 8817 from HGNC:3674. The FGF-18 polypeptides provided herein may also be encoded by a codon-optimized sequence of any one of the sequences provided herein or otherwise known to the ordinarily skilled artisan.

In one embodiment, the FGF-18 polypeptide may be fused with another protein or a fragment of another protein such as, for example, the fragment crystallizable region of an antibody to facilitate optimal residence time and alternative clearance mechanisms. The genetic construct may therefore encode a fusion protein combining FGF-18 polypeptide as provided herein and at least one functional element of another protein. In one embodiment, the fusion partner may be at least a part of an Fc fragment of an antibody, at least a part of a cytokine, at least a part of a growth factor, at least a part of a growth factor receptor, at least a part of a molecule that increases the residence time of a growth factor, or at least a part of a molecule that increases binding affinity to a receptor.

In one embodiment, the nucleic acid encodes the FGF-18 gene and at least one auxiliary or regulatory sequence that facilitates expression of the FGF-18 polypeptide. Such regulatory sequences may include one or more introns from a FGF-18 gene or other human genes, post translational regulatory elements, such as the WPRE or oPRE (e.g., OPRE, WPREmut6, orWPREmutl), genetic sequences encoding the polyA signal, transcription initiation complex binding sequences, protein binding sequences (which bind DNA-binding proteins such as TATA-binding proteins, GATA1, Zn-finger proteins, helicases, nickases, or nucleases, single-stranded binding proteins, transcription initiation complex proteins, or other proteins that can interact with DNA), enhancer sequences, distal and proximal enhancer elements, insulating sequences, the Kozak sequence, termination signals, internal ribosome entry sites, or any other genetic sequence that affects transcription, replication, translation, insertion into the genome, recombination, persistence, level of expression, nuclear translocation, or entry into the cell or a given cell compartment.

The therapeutic construct may be delivered locally, such as to an osteoarthritic or pre-arthritic joint, by a local injection or an intra-articular injection. In one embodiment, the therapeutic construct is delivered to at least some cells of a joint. The cells may be any one of the cells provided herein. In another embodiment, the therapeutic construct is in a formulation that facilitates delivery of the FGF-18 gene and/or other sequences to at least some cells of the joint. Such cells may be any of the cells provided herein.

The therapeutic formulation may contain delivery vectors to deliver the FGF-18 gene and/or other sequences to the cells, target tissues, or desired cellular compartments. In one embodiment, the genetic construct can be delivered by a viral, lipid-based, polymer-based, or hybrid carrier. Some specific viral vectors that can be used include one or more of an adeno-associated virus, an adenovirus, lentivirus, herpes simplex virus, pox virus, measles virus, alphavirus, mimivirus, or other enveloped or non-enveloped virus or functional element thereof. In another embodiment, the viral carrier is a viral vector engineered as synthetic recombinant viral vector or chemically modified post assembly of the capsid. The viral vector can be synthetic, semi-synthetic, engineered, or contain a hybrid protein, or fully hybrid capsid. The formulation of viral capsids may contain empty or full capsids, or capsids containing different sequences, or may be comprised of different viral strains, species, families, or genus, and may be at least 5% full, containing the genetic construct of interest or up to 100% full, containing the genetic construct of interest.

In another embodiment, a viral carrier, natural or synthetic capsid, lipid nanoparticle (LNP), liposome, polymeric carrier, or other non-viral carrier may be coupled to one or more ligands to enhance functionality, selectivity, specificity, residence time, or other physical or chemical property of the capsid or capsid cargo. As used herein, “couple” or “coupled” or “coupling” (and the like) means to chemically associate one entity (for example a ligand) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In one embodiment, encapsulation is a form of coupling. In another embodiment, the coupling is by conjugation via a direct linkage, such as covalent conjugation.

In another embodiment, the genetic construct encoding the FGF-18 gene or other sequences may be formulated in polymeric carrier, such as a solid polymeric carrier, polyelectrolytic complex comprising at least one polymer and the nucleic acid, or multiple polymers and the nucleic acid. The polymeric carrier may be comprised, at least in part, of cationic polymers, anionic polymers, amphiphilic polymers, non-ionic polymers, or polymers that can vary the overall charge from positive to negative, or from neutral to positive, or from neutral to negative as a function of ranging physiological pH or as a function of the changing pH between the original formulation and the physiologic conditions. In one embodiment, the polymeric carrier may be comprised, at least in part, of chitosan, polyethyleneimine, or a polypeptide with an overall positive charge, or combination of one or more of the aforementioned polymers.

In one embodiment, the genetic construct encoding the FGF-18 gene and/or other sequences is formulated in at least a single class of lipid nanoparticles, liposomes, or lipid emulsion with or without the use of ligands to enhance targeting, improve bioavailability, increase residence time, or facilitate more optimal clearance. In one embodiment, the lipid carrier can contain up to 60% by molar ratio cholesterol, or up to 80% by molar ratio a cationic or ionizable lipid. In another embodiment, the lipid carrier can be comprised, at least in part, of glycerides, polyglyceryls, or polyoxylglycerides, or generally be an oil/water nanoemulsion, oil/water microemulsion, nanocapsule, or a self-nanoemulsifying or self-microemulsifying system. In another embodiment, the lipid carrier is a micelle, lipid-polymer hybrid, or is at least in part comprised of a biopolymer or a biomimetic. In one embodiment, lipid carriers contain peptides, proteins, polysaccharides, small molecules, or combinations thereof for targeting, increased uptake, increased nuclear delivery of DNA payload or increased in vivo residence time, or to generally modify one or more physical, chemical, or biological properties of the lipid carrier specifically or one or more elements of the formulation in general.

In one embodiment, the administered dose is calculated based on the surface area of the joint, which may be estimated (e.g., from an MRI imaging scan). Alternatively, the dose can be estimated from the volume of articular cartilage, either nominal (as defined by a healthy state) or at the time of treatment. Yet alternatively, the dose can be estimated from a correlate of the surface area or volume of articular cartilage, such as age, disease state, weight, and species undergoing treatment.

In one embodiment, the therapeutic construct is administered as a function of the joint surface area. For example, the therapeutic construct may be administered such that between 1.2×10² genome copies/cm² of joint surface area to 6.1×10¹¹ genome copies/cm² of joint surface area (or correlate) are administered. As another example, the therapeutic construct may be administered such that between 4.5×10³ genome copies/cm² of joint surface area to 2.3×10¹⁰ genome copies/cm² of joint surface area (or correlate) are administered.

Alternatively, the therapeutic construct may be administered at a dose that is calculated using an average joint size for a given species in relation to weight or using an average joint size for a given species in relation to age or using an average joint size for a given species in relation to weight and age. In one embodiment, the therapeutic construct is administered at a dose of 2×10⁴ genome copies/kg to 6.3×10¹³ genome copies/kg or via a joint surface area correlate of 3.8×10⁴ genome copies/kg to 4.7×10¹³ genome copies/kg in humans or via a joint surface area correlate of 4.5×10\hat{ }5 genome copies/kg to 6.3×10¹³ genome copies/kg in horses or via a joint surface area correlate of 3.8×10⁴ genome copies/kg to 1.2×10¹³ genome copies/kg in dogs or via a joint surface area correlate of 2×10⁴ genome copies/kg to 4.3×10¹² genome copies/kg in cats.

It should be appreciated that the genetic constructs provided herein may be administered to humans or other mammals, as appropriate.

In one embodiment, the therapeutic construct is administered as a correlate of joint surface area to large or small joints that can be generally defined as knee, hip, shoulder, or elbow, or joints approximately comparable to knee, hip, shoulder, or elbow in size, or joints at the extremities, respectively. In one embodiment, the dose is administered via a joint surface area correlate of 5×10⁷ genome copies/knee, hip, or shoulder joint to 2×10¹⁴ genome copies/knee, hip, or shoulder joint in humans or via a joint surface area correlate of 5×10⁷ genome copies/knee, hip, or shoulder joint to 5×10¹³ genome copies/knee, hip, or shoulder joint in horses or via a joint surface area correlate of 1×10⁵ genome copies/knee, hip, or shoulder joint to 1×10¹ genome copies/knee, hip, or shoulder joint in dogs or via a joint surface area correlate of 5×10⁴ genome copies/knee, hip, or shoulder joint to 7×10¹¹ genome copies/knee, hip, or shoulder joint in cats or via a joint surface area correlate of 4×10⁷ genome copies/knee, hip, or shoulder joint to 2×10¹⁴ genome copies/knee, hip, or shoulder joint. In another embodiment, a dose of the therapeutic construct is administered via a volume or surface area correlate of 5×10⁸ genome copies/knee, hip, or shoulder joint to 1×10¹³ genome copies/knee, hip, or shoulder joint or 5×10⁸ genome copies/knee, hip, or shoulder joint to 8×10¹² genome copies/knee, hip, or shoulder joint.

Any one of the methods provided herein may include one or more steps of determining a dose based on the concepts provided herein. In one aspect, a method comprising one or more steps of determining a dose based on the concepts provided herein is provided. Any one of the methods provided herein can comprise administering a genetic construct or composition comprising the genetic construct at a dose provided herein or determined by a method as provided herein. Any one of the compositions provided herein can comprise a genetic construct at a dose provided herein or determined by a method as provided herein.

In one embodiment, adjustments can be made to the dose based on age, sex, and disease severity by utilizing average multiples of the cartilage thickness, volume, or surface area for the respective species, age, sex, and disease severity. Any one of the methods provided herein can include a step of such an adjustment. Any one of the methods provided herein can comprise administering a genetic construct or composition comprising the genetic construct at a dose provided herein or determined by a method comprising a step of adjustment. Any one of the compositions provided herein can comprise a genetic construct at a dose provided herein or determined by a method comprising a step of adjustment.

Compositions according to the invention can comprise pharmaceutically acceptable excipients, such as preservatives, buffers, saline, or phosphate buffered saline. “Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. The compositions may be made using conventional pharmaceutical manufacturing and compounding techniques to arrive at useful dosage forms. In an embodiment, compositions are suspended in sterile saline solution for injection together with a preservative.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. It is to be understood that the compositions of the invention can be made in any suitable manner, and the invention is in no way limited to compositions that can be produced using the methods described herein. Selection of an appropriate method of manufacture may require attention to the desired functionalities.

In some embodiments, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection. In some embodiments, the compositions may be lyophilized and stored in suspension or as lyophilized powder depending on the formulation strategy for extended periods without losing activity. The compositions referred to herein may be manufactured and prepared for administration using conventional methods.

The compositions or gene construct(s) provided herein may be delivered without limitation via a drug delivery device such as a prefilled syringe, or may be stored in an ampoule, vial, form-fill-seal, or blow-fill-seal container for administration via a secondary suitable means thereafter. Kits comprising any one of the compositions or gene construct(s) provided herein are also provided.

It should be understood that within the scope of this invention the formulation, materials, genetic constructs, sequences, biological and chemical compositions, or methods of use may be varied by one skilled in the art, to the extent that they perform the desired function as provided herein. Various parts, components or characteristics may be used in combination, with or without modification by someone skilled in the art to achieve the desired functionality as provided herein.

Moreover, all individual features and methods of use described herein, and each and every combination of two or more of such features and methods of use, are included within the scope of the present invention provided that these features and methods of use in such a combination are not mutually inconsistent. It is understood that certain portions or combinations of such portions can be varied by someone trained in the art while still achieving the goals of the invention.

Finally, it is understood that the specific ranges provided in the current invention are not restrictive and are for example purposes only, values outside of the specified ranges may be used to achieve the goal of the invention without modification to the proposed mechanistic principals.

### EXAMPLES

### Example 1

AAV2 vector encoding hFGF18 gene without codon optimization driven by a cartilage specific promoter Col2a1 regulated by WPRE post-translational response element. Thus, in one embodiment, the therapeutic may be an AAV2 vector encoding hFGF18 gene without codon optimization and driven by a cartilage specific promoter Col2a1 and, optionally, regulated by WPRE post-translational response element. This therapeutic may be delivered intra-articularly. Transduction of chondrocytes can result and provide autocrine and paracrine cues for cartilage repair. The cartilage-specific promoter Col2a1 can serves as a means to increase specificity of the treatment that is administered locally to a joint.

(SEQ ID NO: 1)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTTCTAGACAACTTTGTATAGAAA
AGTTGCCTCACAAAGCCGAGTCCAGCTGGAGCCACCTCCAGACTCTCTGGCCAG
AGCCTCAGAGTGGCCAACAGTCCCTGGCCAGCGGTTGCTCTATCCAGGCTAAGG
GCACCCACTCCCCTGGAGATTCCTGAACCTGGGCCAGGAAGAGCCGAACTAGAC
AAGCGTCTCCAATCCATCCCGTTTTGTCCCCGGGGTTGTCTGCAGTCCCGTTAGTC
CCTTCCCTGGCCTGTTCGCCCCTTAGACCTCCACACAGGTCTCCCTCTGTGTAGGA
ATTCACCAGACCCTGTCCTAGCCACCCGCTCAGTTCCCCCTTCTACCTAGGCCTTT
TCTAGCTAGTTGGATGGGGGATGGGTTAGGGAGGCTGTGGTCCCCGAGACTCCA
GGTGGGAGCTCACGGGCAGGTACTCCGCAAAGGAGCTGGAAGGCAGGTCTGGA
AAACTGTCCCCCAGATTTAGGATTCTGGACAACGTCCATCTGCTCATGCTTTGGT
CCCCCACGCACCTCCCCGCCTCCTAAATTCCCCATCCCCACCTTTCTTGCTCCTTT
CTGTTGCTTCGTCCTCTCTGCCTTGGGTCTAAAACTCCAGGCTTATGCCTCTGCAA
ACAACCCCCTCCCTTCTAACCCCAGCAGAACTCCGAGGAAAGGGGCCCGATGCT
CCCCCCCTTCCCGCCTGTGGTTAGAGGGGGCAGTGTGGCAGTCCCAAGTGGGGG
CGACCGGAGGCTGTCTCGGTGCCCCGCCCGATCAGGCCACTGGGCACATTGGGG
GCGGGAAGCTGGGCTCACGAAAGGGGCGACTGGCCTTGGCAGGTGTGGGCTCTG
GTCCGGCCTGGGGGGCTCCGGGGGCGGGGTCTCAGGTTACAGCCCCGCGGGGG
GCTAGGGGGCGGCCCGCGGTTTGGGCCGGTTTGCCAGCCTTTGGAGCGACCGGG
AGCATATAACTGGAGCCTCTGCCGGGGGAAGACGCAGAGCGCCGCTGGGCTGCC
GGGTCTCCTGCCTCCTCCTGCTCCTAGGGCCTCCTGCATGAGGGAGCGGTAGAGA
CCAAGTTTGTACAAAAAAGCAGGCTGCCACCATGTATTCAGCGCCCTCCGCCTGC
ACTTGCCTGTGTTTACACTTCCTGCTGCTGTGCTTCCAGGTACAGGTGCTGGTTGC
CGAGGAGAACGTGGACTTCCGCATCCACGTGGAGAACCAGACGCGGGCTCGGGA
CGATGTGAGCCGTAAGCAGCTGCGGCTGTACCAGCTCTACAGCCGGACCAGTGG
GAAACACATCCAGGTCCTGGGCCGCAGGATCAGTGCCCGCGGCGAGGATGGGGA
CAAGTATGCCCAGCTCCTAGTGGAGACAGACACCTTCGGTAGTCAAGTCCGGAT
CAAGGGCAAGGAGACGGAATTCTACCTGTGCATGAACCGCAAAGGCAAGCTCGT
GGGGAAGCCCGATGGCACCAGCAAGGAGTGTGTGTTCATCGAGAAGGTTCTGGA
GAACAACTACACGGCCCTGATGTCGGCTAAGTACTCCGGCTGGTACGTGGGCTTC
ACCAAGAAGGGGCGGCCGCGGAAGGGCCCCAAGACCCGGGAGAACCAGCAGGA
CGTGCATTTCATGAAGCGCTACCCCAAGGGGCAGCCGGAGCTTCAGAAGCCCTT
CAAGTACACGACGGTGACCAAGAGGTCCCGTCGGATCCGGCCCACACACCCTGC
CTAGACCCAGCTTTCTTGTACAAAGTGGGAATTCCGATAATCAACCTCTGGATTA
CAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTC
ATTTTCTCCTCCTTGTATAAATCCTGGTTGCTGTCTCTTTATGAGGAGTTGTGGCC
CGTTGTCAGGCAACGTGGCGTGGTGTGCACTGTGTTTGCTGACGCAACCCCCACT
GGTTGGGGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGACAGGG
GCTCGGCTGTTGGGCACTGACAATTCCGTGGTGTTGTCGGGGAAGCTGACGTCCT
TTCCATGGCTGCTCGCCTGTGTTGCCACCTGGATTCTGCGCGGGACGTCCTTCTGC
TACGTCCCTTCGGCCCTCAATCCAGCGGACCTTCCTTCCCGCGGCCTGCTGCCGG
CTCTGCGGCCTCTTCCGCGTCTTCGCCTTCGCCCTCAGACGAGTCGGATCTCCCTT
TGGGCCGCCTCCCCGCATCGGGAATTCCTAGAGCTCGCTGATCAGCCTCGACTGT
GCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAA
GGGGGAGGATTGGGAAGAGAATAGCAGGCATGCTGGGGAGGGCCGCAGGAACC
CCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGC
GAGCGAGCGCGCAGCTGCCTGCAGG

### Example 2

A CMV-promoter driven AAV-delivered, codon-optimized hFGF-18-encoding construct with an oPRE post-translational response element. Thus, in another embodiment, the therapeutic may be an AAV vector encoding a CMV-promoter and codon-optimized hFGF-18. The therapeutic may be delivered intra-articularly and can provide transduction efficiency locally in the joint and can drive a high-level of expression relative to the number of viral particles administered.

(SEQ ID NO: 2)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTTCTAGACAACTTTGTATAGAAA
AGTTGTAGTTATTAATAGTAATCAATTACGGGGTCATTAGTTCATAGCCCATATA
TGGAGTTCCGCGTTACATAACTTACGGTAAATGGCCCGCCTGGCTGACCGCCCAA
CGACCCCCGCCCATTGACGTCAATAATGACGTATGTTCCCATAGTAACGCCAATA
GGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGG
TAAATGGCCCGCCTGGCATTATGCCCAGTACATGACCTTATGGGACTTTCCTACT
TGGCAGTACATCTACGTATTAGTCATCGCTATTACCATGGTGATGCGGTTTTGGC
AGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGG
TGGGAGGTCTATATAAGCAGAGCTGGTTTAGTGAACCGTCAGATCCAAGTTTGTA
CAAAAAAGCAGGCTGCCACCATGTACAGCGCTCCAAGCGCCTGCACATGCCTGT
GTCTGCACTTCCTGCTGCTGTGCTTCCAGGTGCAGGTGCTGGTGGCCGAGGAGAA
CGTGGATTTTAGGATCCACGTGGAAAACCAGACCAGAGCCAGAGATGATGTGAG
CAGGAAGCAGCTGCGGCTGTACCAGCTGTATTCAAGGACCAGCGGCAAACACAT
CCAGGTGCTGGGACGGAGGATCTCCGCCAGAGGGGAGGACGGTGACAAGTACG
CCCAGCTGCTTGTGGAGACTGATACATTTGGCTCCCAGGTGAGAATCAAGGGCA
AGGAGACCGAGTTTTACCTGTGCATGAACAGGAAAGGCAAGCTGGTGGGCAAGC
CAGACGGGACCAGCAAGGAGTGCGTGTTCATCGAGAAGGTGCTGGAGAACAACT
ATACCGCCCTGATGTCCGCCAAGTACTCTGGCTGGTACGTGGGATTCACAAAGAA
GGGAAGGCCTCGCAAGGGCCCTAAGACCCGGGAGAACCAGCAGGATGTCCACTT
CATGAAGAGATACCCTAAGGGACAGCCCGAACTGCAGAAGCCCTTCAAGTACAC
AACTGTGACAAAGAGAAGCAGGAGAATCAGACCAACTCACCCCGCCTGAACCCA
GCTTTCTTGTACAAAGTGGGAATTCGAGCATCTTACCGCCATTTATACCCATATTT
GTTCTGTTTTTCTTGATTTGGGTATACATTTAAATGTTAATAAAACAAAATGGTGG
GGCAATCATTTACATTTTTAGGGATATGTAATTACTAGTTCAGGTGTATTGCCAC
AAGACAAACATGTTAAGAAACTTTCCCGTTATTTACGCTCTGTTCCTGTTAATCA
ACCTCTGGATTACAAAATTTGTGAAAGATTGACTGATATTCTTAACTATGTTGCTC
CTTTTACGCTGTGTGGATATGCTGCTTTATAGCCTCTGTATCTAGCTATTGCTTCC
CGTACGGCTTTCGTTTTCTCCTCCTTGTATAAATCCTGGTTGCTGTCTCTTTTAGAG
GAGTTGTGGCCCGTTGTCCGTCAACGTGGCGTGGTGTGCTCTGTGTTTGCTGACG
CAACCCCCACTGGCTGGGGCATTGCCACCACCTGTCAACTCCTTTCTGGGACTTT
CGCTTTCCCCCTCCCGATCGCCACGGCAGAACTCATCGCCGCCTGCCTTGCCCGC
TGCTGGACAGGGGCTAGGTTGCTGGGCACTGATAATTCCGTGGTGTTGTCGAATT
CCTAGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCT
GGGGGGTGGGGTGGGGCAGGACAGCAAGGGGGAGGATTGGGAAGAGAATAGCA
GGCATGCTGGGGAGGGCCGCAGGAACCCCTAGTGATGGAGTTGGCCACTCCCTC
TCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGCCTGCAGG

### Example 3

A tetracycline inducible promoter activated construct delivering hFGF18 with a WPRE post-translational response element. It may be desirable to induce periodic expression or activate expression during periods of increased pain or following cartilage loss. In one embodiment, the therapeutic comprises a tetracycline inducible promoter to regulate expression of hFGF-18. This therapeutic may be delivered intra-articularly allowing for temporal control of expression locally in the joint.

(SEQ ID NO: 3)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTTCTAGACAACTTTGTATAGAAA
AGTTGGGTACCGAGCTCGACTTTCACTTTTCTCTATCACTGATAGGGAGTGGTAA
ACTCGACTTTCACTTTTCTCTATCACTGATAGGGAGTGGTAAACTCGACTTTCACT
TTTCTCTATCACTGATAGGGAGTGGTAAACTCGACTTTCACTTTTCTCTATCACTG
ATAGGGAGTGGTAAACTCGACTTTCACTTTTCTCTATCACTGATAGGGAGTGGTA
AACTCGACTTTCACTTTTCTCTATCACTGATAGGGAGTGGTAAACTCGACTTTCAC
TTTTCTCTATCACTGATAGGGAGTGGTAAACTCGACCTATATAAGCAGAGCTCGT
TTAGTGAACCGTCAGATCGCCTGGAGACGCCATCCACGCTGTTTTGACCTCCATA
GAAGACACCGGGACCGATCCAGCCTCCGCGGCCCCGAATTGCAAGTTTGTACAA
AAAAGCAGGCTGCCACCATGTATTCAGCGCCCTCCGCCTGCACTTGCCTGTGTTT
ACACTTCCTGCTGCTGTGCTTCCAGGTACAGGTGCTGGTTGCCGAGGAGAACGTG
GACTTCCGCATCCACGTGGAGAACCAGACGCGGGCTCGGGACGATGTGAGCCGT
AAGCAGCTGCGGCTGTACCAGCTCTACAGCCGGACCAGTGGGAAACACATCCAG
GTCCTGGGCCGCAGGATCAGTGCCCGCGGCGAGGATGGGGACAAGTATGCCCAG
CTCCTAGTGGAGACAGACACCTTCGGTAGTCAAGTCCGGATCAAGGGCAAGGAG
ACGGAATTCTACCTGTGCATGAACCGCAAAGGCAAGCTCGTGGGGAAGCCCGAT
GGCACCAGCAAGGAGTGTGTGTTCATCGAGAAGGTTCTGGAGAACAACTACACG
GCCCTGATGTCGGCTAAGTACTCCGGCTGGTACGTGGGCTTCACCAAGAAGGGG
CGGCCGCGGAAGGGCCCCAAGACCCGGGAGAACCAGCAGGACGTGCATTTCATG
AAGCGCTACCCCAAGGGGCAGCCGGAGCTTCAGAAGCCCTTCAAGTACACGACG
GTGACCAAGAGGTCCCGTCGGATCCGGCCCACACACCCTGCCTAGACCCAGCTTT
CTTGTACAAAGTGGGAATTCCGATAATCAACCTCTGGATTACAAAATTTGTGAAA
GATTGACTGGTATTCTTAACTATGTTGCTCCTTTTACGCTATGTGGATACGCTGCT
TTAATGCCTTTGTATCATGCTATTGCTTCCCGTATGGCTTTCATTTTCTCCTCCTTG
TATAAATCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGGCATTGC
CACCACCTGTCAGCTCCTTTCCGGGACTTTCGCTTTCCCCCTCCCTATTGCCACGG
CGGAACTCATCGCCGCCTGCCTTGCCCGCTGCTGGACAGGGGCTCGGCTGTTGGG
CACTGACAATTCCGTGGTGTTGTCGGGGAAGCTGACGTCCTTTCCATGGCTGCTC
GCCTGTGTTGCCACCTGGATTCTGCGCGGGACGTCCTTCTGCTACGTCCCTTCGGC
CCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCC
GCATCGGGAATTCCTAGAGCTCGCTGATCAGCCTCGACTGTGCCTTCTAGTTGCC
AGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGG
AAGAGAATAGCAGGCATGCTGGGGAGGGCCGCAGGAACCCCTAGTGATGGAGTT
GGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGC
TGCCTGCAGG

### Example 4

A self-complimentary AAV vector delivering hFGF18 with codon optimization under the regulation of a tissue specific promoter and containing an SV40 late polyA sequence. In another embodiment, the therapeutic comprises a self-complimentary AAV vector delivering hFGF18 with codon optimization under the regulation of a tissue-specific promoter and, optionally, containing an SV40 late polyA sequence. The therapeutic may be delivered intra-articularly. The therapeutic can be used to optimize the level of expression of FGF-18 locally in the joint relative to the number of viral particles delivered, with a strong polyA signal that stops expression at the end of the coding sequence and stabilizes the messenger RNA.

(SEQ ID NO: 4)
GGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGA
GAGGGAGTGGCCAACTCCATCACTAGGGGTTCCTGGAGGGGTGGAGTCGTGACC
TAGGCAACTTTGTATAGAAAAGTTGCTCCTGGGCTCCCAAATGCTGGCTCCAGTC
AAGGCTTCAGTCGTGGTCCTCTGGGAGCATCCAGGCTGGGGTGGTTTGGCAAGG
GGCTTACTAGACCATCATGTGCAGAGAGCAGCAGAGCCCCTTGTACAAAAACCA
GGACCCCTGGGTTCCCTGTGGATAGGATGAGCTGCCAAGGAGATGTCACTCTTGG
CACCCTCCAAGAATCATTACGGCCGGGTGCAGTGGCTCATGCTTGAAATCCCAGC
ACTTTGGGAGGCCGAGGCGGGTGAATCACCTGAGGTCAGGAGTTCGAGACCAGC
CTGGCCAACATGGTGAAATCCTGTCTCTACTAAAAATACAAAATTAGCCAGGCAT
GGTGGCATGTGCCTGTAATCCCAGCTACTCGGGAGGCTGAGGCAGGAGAATCAC
TTGAACCCGGGAGGCGGAGGTTGCAGTCAGCCAAGATTGCGCCACTGTACTCCA
GCCTGGGCAACAAGAGTGAAACTCCATCTCAAAAAAAAATACAGAATTAGCCAG
GCATGGTGGCGTGTGCCTGTAGTCCCAGCTACTCAAGAGGCTGAGGCAGGAGAA
TCACTTGAACCTGGGAGGCAGAGGTTGCAGTGAGCCGAGATCGCACCATTGCAC
TCCTGCCTGGGCGACAGAGCGAGACTCCGTCTCAAAAAAAAAGAACCGCGAGCA
CTTTGTTTCTCTTGCTTCTTCCACTTCACTCTTCTTCAGCCAGGGCAGCCAAAGGC
AGGATGGGTGCTTCCCGGATCCACCAGAGGGGGAAAGGGGTTGGGCGTGTATGG
GGGCAGAACTTTTCCCTAGTGAGAGCCTTGGTGAGGTCTGCTGGAGGCACACTG
GGGACACAGGCAAGCATCAGGTGGTCCCAACTTCCCACCCCCCTCCCATAAACG
GCACTTGGCTCAGTCTCCCTCTGCCCACCTTGTCACCATGGAGCTGTGGCATTCA
AGCATCCAAATGCCCTGCTTCTGTCAGGCCTGCCCCGTCGGGTGCTTTGTTTACTT
GTCAGGTTGGGCAGATGGTCTCAAGCCCTGGTTGGTGGGTGGGTGAGTGAGGAG
ACAGGTTAGACAGGGGAAGGTCCCAGGACATGTCTTCCCTCTCTGGGCTGACCTG
AGGCAGTGGAGGCTCTCAGGTGTGGGATGGGGTTTTCAGGCTGGGATGTTCTGTA
CCGTAGAGGATAGGCCTTCCTATTACTTATCGGAGCACTACGGGAGGGCAGGTC
CCTCCCCAGGGTGTTTAACACTGGAGGCTGCAGGGTCAGGAGGAGAATCGTGGG
GCCAGGAGGGCAGAGGCACACTCCATCTTCGTGCTCCTCACAGGCCCTGCCTCCC
TGCCTGCTAAGGACACAGGGAAGGGGGTCCCCACCTCAGTGCCTGCCTCCCTTCC
CTGTGCCTGTGTACCTGGCAGTCACAGCCACCTGGCGTGTCCCAGAAACCAACCG
GCTGACCTCATCTCCTGCCCGGCCCCACCTCCATTGGCTTTGGCTTTTGGCGTTTG
TGCTGCCCGACCCTTTCTCCTGTCCGGATGCGCAGGGCAGGGCCTGAGCCGTCGA
GCTGCACCCACAGCAGGCTGCCTTTGGTGACTCACCGGGTGAACGGGGGCATTG
CGAGGCATCCCCTCCCTGGGTTTGGCTCCTGCCCACGGGGCTGACAGTAGAAATC
ACAGGCTGTGAGACAGCTGGAGCCCAGCTCTGCTTGAACCTATTTTAGGTCTCTG
ATCCCCGCTTCCTCTTTAGACTCCCCTAGAGCTCAGCCAGTGCTCAACCTGAGGC
TGGGGGTCTCTGAGGAAGAGTGAGTTGGAGCTGAGGGGTCTGGGGCTGTCCCCT
GAGAGAGGGGCCAGAGGCAGTGTCAAGAGCCGGGCAGTCTGATTGTGGCTCACC
CTCCATCACTCCCAGGGGCCCCTGGCCCAGCAGCCGCAGCTCCCAACCACAATAT
CCTTTGGGGTTTGGCCTACGGAGCTGGGGCGGATGACCCCCAAATAGCCCTGGC
AGATTCCCCCTAGACCCGCCCGCACCATGGTCAGGCATGCCCCTCCTCATCGCTG
GGCACAGCCCAGAGGGTATAAACAGTGCTGGAGGCTGGCGGGGCAGGCCAGCT
GAGTCCTGAGCAGCAGCCCAGCGCAGCCACCGAGACACCCAAGTTTGTACAAAA
AAGCAGGCTGCCACCATGTACAGCGCTCCAAGCGCCTGCACATGCCTGTGTCTGC
ACTTCCTGCTGCTGTGCTTCCAGGTGCAGGTGCTGGTGGCCGAGGAGAACGTGGA
TTTTAGGATCCACGTGGAAAACCAGACCAGAGCCAGAGATGATGTGAGCAGGAA
GCAGCTGCGGCTGTACCAGCTGTATTCAAGGACCAGCGGCAAACACATCCAGGT
GCTGGGACGGAGGATCTCCGCCAGAGGGGAGGACGGTGACAAGTACGCCCAGCT
GCTTGTGGAGACTGATACATTTGGCTCCCAGGTGAGAATCAAGGGCAAGGAGAC
CGAGTTTTACCTGTGCATGAACAGGAAAGGCAAGCTGGTGGGCAAGCCAGACGG
GACCAGCAAGGAGTGCGTGTTCATCGAGAAGGTGCTGGAGAACAACTATACCGC
CCTGATGTCCGCCAAGTACTCTGGCTGGTACGTGGGATTCACAAAGAAGGGAAG
GCCTCGCAAGGGCCCTAAGACCCGGGAGAACCAGCAGGATGTCCACTTCATGAA
GAGATACCCTAAGGGACAGCCCGAACTGCAGAAGCCCTTCAAGTACACAACTGT
GACAAAGAGAAGCAGGAGAATCAGACCAACTCACCCCGCCTGAACCCAGCTTTC
TTGTACAAAGTGGTGATGGCCGGCCGCTTCGAGCAGACATGATAAGATACATTG
ATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGTT
AACAACAACAATTGCATTCATTTTATGTTTCAGGTTCAGGGGGAGGTGTGGGAGG
TTTTTTAAAGCAAGTAAAACCTCTACAAATGTGGTAACTAGTCCACTCCCTCTCT
GCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGAGAGGGA

### Example 5

An AAV vector delivering dgFGF18 without codon optimization under the regulation of a CBh promoter and containing an oPRE post-translational regulatory element.

(SEQ ID NO: 5)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTTCTAGACAACTTTGTATAGAAA
AGTTGCGTTACATAACTTACGGTAAATGGCCCGCCTGGCTGACCGCCCAACGACC
CCCGCCCATTGACGTCAATAGTAACGCCAATAGGGACTTTCCATTGACGTCAATG
GGTGGAGTATTTACGGTAAACTGCCCACTTGGCAGTACATCAAGTGTATCATATG
CCAAGTACGCCCCCTATTGACGTCAATGACGGTAAATGGCCCGCCTGGCATTGTG
CCCAGTACATGACCTTATGGGACTTTCCTACTTGGCAGTACATCTACGTATTAGT
CATCGCTATTACCATGGTCGAGGTGAGCCCCACGTTCTGCTTCACTCTCCCCATCT
CCCCCCCCTCCCCACCCCCAATTTTGTATTTATTTATTTTTTAATTATTTTGTGCAG
CGATGGGGGCGGGGGGGGGGGGGGGGCGCGCGCCAGGCGGGGGGGGCGGGGC
GAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAA
AAAGCGAAGCGCGCGGCGGGCGGGAGTCGCTGCGCGCTGCCTTCGCCCCGTGCC
CCGCTCCGCCGCCGCCTCGCGCCGCCCGCCCCGGCTCTGACTGACCGCGTTACTC
CCACAGGTGAGCGGGCGGGACGGCCCTTCTCCTCCGGGCTGTAATTAGCTGAGC
AAGAGGTAAGGGTTTAAGGGATGGTTGGTTGGTGGGGTATTAATGTTTAATTACC
TGGAGCACCTGCCTGAAATCACTTTTTTTCAGGTTGGCAAGTTTGTACAAAAAAG
CAGGCTGCCACCATGTATTCAGCGCCCTCCGCCTGCACTTGCCTGTGTTTACACTT
CCTGCTGCTGTGCTTCCAGGTACAGGTGCTGGCGGCCGAAGAGAACGTGGACTTC
CGCATCCACGTGGAGAACCAGACGCGGGCTCGGGACGATGTGAGCCGTAAGCAG
CTGCGGCTGTACCAGCTCTACAGCCGGACCAGTGGGAAGCACATCCAGGTCCTG
GGCCGCAGGATCAGTGCCCGCGGCGAGGACGGGGACAAGTATGCCCAGCTCCTA
GTGGAGACAGACACCTTCGGTAGTCAAGTCCGGATCAAGGGCAAGGAGACGGA
ATTCTACCTATGTATGAACCGGAAAGGCAAGCTCGTGGGGAAGCCCGATGGCAC
CAGCAAGGAGTGTGTATTCATTGAGAAAGTCCTGGAGAACAACTATACAGCCCT
GATGTCCGCCAAGTACTCTGGCTGGTACGTGGGCTTCACCAAGAAGGGGCGGCC
CCGGAAGGGTCCCAAGACCCGGGAGAACCAGCAGGACGTGCACTTCATGAAGCG
CTACCCCAAGGGACAGGCAGAGCTGCAGAAGCCCTTCAAGTACACCACGGTGAC
CAAGAGGTCCCGGCGGATCCGCCCCACGCACCCCGGCTAGACCCAGCTTTCTTGT
ACAAAGTGGGAATTCGAGCATCTTACCGCCATTTATACCCATATTTGTTCTGTTTT
TCTTGATTTGGGTATACATTTAAATGTTAATAAAACAAAATGGTGGGGCAATCAT
TTACATTTTTAGGGATATGTAATTACTAGTTCAGGTGTATTGCCACAAGACAAAC
ATGTTAAGAAACTTTCCCGTTATTTACGCTCTGTTCCTGTTAATCAACCTCTGGAT
TACAAAATTTGTGAAAGATTGACTGATATTCTTAACTATGTTGCTCCTTTTACGCT
GTGTGGATATGCTGCTTTATAGCCTCTGTATCTAGCTATTGCTTCCCGTACGGCTT
TCGTTTTCTCCTCCTTGTATAAATCCTGGTTGCTGTCTCTTTTAGAGGAGTTGTGG
CCCGTTGTCCGTCAACGTGGCGTGGTGTGCTCTGTGTTTGCTGACGCAACCCCCA
CTGGCTGGGGCATTGCCACCACCTGTCAACTCCTTTCTGGGACTTTCGCTTTCCCC
CTCCCGATCGCCACGGCAGAACTCATCGCCGCCTGCCTTGCCCGCTGCTGGACAG
GGGCTAGGTTGCTGGGCACTGATAATTCCGTGGTGTTGTCGAATTCCTAGAGCTC
GCTGATCAGCCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGG
GGTGGGGCAGGACAGCAAGGGGGAGGATTGGGAAGAGAATAGCAGGCATGCTG
GGGAGGGCCGCAGGAACCCCTAGTGATGGAGTTGGCCACTCCCTCTCTGCGCGC
TCGCTCGCTCACTGAGGCCGGGCGACCAAAGGTCGCCCGACGCCCGGGCTTTGC
CCGGGCGGCCTCAGTGAGCGAGCGAGCGCGCAGCTGCCTGCAGGGGCGCCTGAT
GCGGTATTTTCTCCTTACGCATCTGTGCGGTATTTCACACCGCATACGTCAAAGC
AACCATAGTACGCGCCCTGTAGCGGCGCATTAAGCGCGGCGGGGGTGGTGGTTA
CGCGCAGCGTGACCGCTACACTTGCCAGCGCCTTAGCGCCCGCTCCTTTCGCTTT
CTTCCCTTCCTTTCTCGCCACGTTCGCCGGCTTTCCCCGTCAAGCTCTAAATCGGG
GGCTCCCTTTAGGGTTCCGATTTAGTGCTTTACGGCACCTCGACCCCAAAAAACT
TGATTTGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTGGAA
CAACACTCAACTCTATCTCGGGCTATTCTTTTGATTTATAAGGGATTTTGCCGATT
TCGGTCTATTGGTTAAAAAATGAGCTGATTTAACAAAAATTTAACGCGAATTTTA
ACAAAATATTAACGTTTACAATTTTATGGTGCACTCTCAGTACAATCTGCTCTGAT
GCCGCATAGTTAAGCCAGCCCCGACACCCGCCAACACCCGCTGACGCGCCCTGA
CGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGACGAAAGG
GCCTCGTGATACGCCTATTTTTATAGGTTAATGTCATGATAATAATGGTTTCTTAG
ACGTCAGGTGGCACTTTTCGGGGAAATGTGCGCGGAACCCCTATTTGTTTATTTTT
CTAAATACATTCAAATATGTATCCGCTCATGAGACAATAACCCTGATAAATGCTT
CAATAATATTGAAAAAGGAAGAGTATGAGTATTCAACATTTCCGTGTCGCCCTTA
TTCCCTTTTTTGCGGCATTTTGCCTTCCTGTTTTTGCTCACCCAGAAACGCTGGTG
AAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAA
TGATGAGCACTTTTAAAGTTCTGCTATGTGGCGCGGTATTATCCCGTATTGACGC
CGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACA
ACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGAC
GAGCGTGACACCACGATGCCTGTAGCAATGGCAACAACGTTGCGCAAACTATTA
ACTGGCGAACTACTTACTCTAGCTTCCCGGCAACAATTAATAGACTGGATGGAGG
CGGATAAAGTTGCAGGACCACTTCTGCGCTCGGCCCTTCCGGCTGGCTGGTTTAT
TGCTGATAAATCTGGAGCCGGTGAGCGTGGAAGCCGCGGTATCATTGCAGCACT
GGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGAT
TAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAA
AAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACC
AACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTC
GTGTCTTACCGGGTTGGACTCAAGACGATAGTTACCGGATAAGGCGCAGCGGTC
GGGCTGAACGGGGGGTTCGTGCACACAGCCCAGCTTGGAGCGAACGACCTACAC
CGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTT
TTGCTCACATGT

### Example 6

An AAV vector delivering ctFGF18 without codon optimization under the regulation of a CAG promoter and containing a WPRE post-translational regulatory element.

(SEQ ID NO: 6)
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTTCTAGACAACTTTGTATAGAAA
AGTTGCTCGACATTGATTATTGACTAGTTATTAATAGTAATCAATTACGGGGTCA
TTAGTTCATAGCCCATATATGGAGTTCCGCGTTACATAACTTACGGTAAATGGCC
CGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTA
CGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCA
CCCCCAATTTTGTATTTATTTATTTTTTAATTATTTTGTGCAGCGATGGGGGGGG
GGGGGGGGGGGGGCGCGCGCCAGGCGGGGCGGGGCGGGGCGAGGGGCGGGGC
GGGGCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCC
GCCTCGCGCCGCCCGCCCCGGCTCTGACTGACCGCGTTACTCCCACAGGTGAGCG
GGCGGGACGGCCCTTCTCCTCCGGGCTGTAATTAGCGCTTGGTTTAATGACGGCT
TGTTTCTTTTCTGTGGCTGCGTGAAAGCCTTGAGGGGCTCCGGGAGGGCCCTTTG
TGCGGGGGGAGCGGCTCGGGGGGTGCGTGCGTGTGTGTGTGCGTGGGGAGCGCC
GCGTGCGGCTCCGCGCTGCCCGGCGGCTGTGAGCGCTGCGGGCGCGGCGCGGGG
CTTTGTGCGCTCCGCAGTGTGCGCGAGGGGAGCGCGGCCGGGGGCGGTGCCCCG
CGGTGCGGGGGGGGCTGCGAGGGGAACAAAGGCTGCGTGCGGGGTGTGTGCGT
GGGGGGGTGAGCAGGGGGTGTGGGCGCGTCGGTCGGGCTGCAACCCCCCCTGCA
CCCCCCTCCCCGAGTTGCTGAGCACGGCCCGGCTTCGGGTGCGGGGCTCCGTACG
GGGCGTGGCGCGGGGCTCGCCGTGCCGGGCGGGGGGTGGCGGCAGGTGGGGGT
GCCGGGCGGGGGGGGCCGCCTCGGGCCGGGGAGGGCTCGGGGGAGGGGCGCG
GCGGCCCCCGGAGCGCCGGCGGCTGTCGAGGCGCGGCGAGCCGCAGCCATTGCC
TTTTATGGTAATCGTGCGAGAGGGCGCAGGGACTTCCTTTGTCCCAAATCTGTGC
GGAGCCGAAATCTGGGAGGCGCCGCCGCACCCCCTCTAGCGGGCGCGGGGCGAA
GCGGTGCGGCGCCGGCAGGAAGGAAATGGGCGGGGAGGGCCTTCGTGCGTCGCC
GCGCCGCCGTCCCCTTCTCCCTCTCCAGCCTCGGGGCTGTCCGCGGGGGGACGGC
TGCCTTCGGGGGGGACGGGGCAGGGCGGGGTTCGGCTTCTGGCGTGTGACCGGC
GGCTCTAGAGCCTCTGCTAACCATGTTCATGCCTTCTTCTTTTTCCTACAGCTCCT
GGGCAACGTGCTGGTTATTGTGCTGTCTCATCATTTTGGCAAAGAATTGCAAGTT
TGTACAAAAAAGCAGGCTGCCACCATGTATTCAGCGCCCTCCGCCTGCACTTGCC
TGTGTTTACACTTCCTGCTGCTGTGCTTCCAGGTACAGGTGCTGGCGGCCGAGGA
GAACGTGGACTTCCGCATCCACGTGGAGAACCAGACGCGGGCTCGGGACGATGT
GAGCCGTAAGCAGCTGCGGCTGTACCAGCTCTACAGCCGGACCAGCGGGAAGCA
CATCCAGGTCCTGGGCCGCAGGATCAGTGCCCGCGGCGAGGACGGGGACAAGTA
TGCCCAGCTCCTAGTGGAGACAGACACCTTCGGTAGTCAAGTCCGGATCAAGGG
CAAGGAGACGGAATTCTACCTGTGTATGAACCGGAAAGGCAAGCTCGTGGGGAA
GCCCGATGGCACCAGCAAGGAGTGTGTGTTCATTGAGAAGGTCCTGGAGAACAA
CTACACAGCCCTGATGTCCGCCAAATACTCTGGCTGGTACGTGGGCTTCACCAAG
AAGGGGCGGCCACGGAAGGGTCCCAAGACCCGGGAGAACCAGCAGGACGTGCA
CTTCATGAAGCGCTACCCCAAGGGACAGGCAGAGCTGCAGAAGCCCTTCAAGTA
CACCACGGTGACCAAGAGGTCCCGGCGGATCCGCCCCACGCACCCAGGCTAGAC
CCAGCTTTCTTGTACAAAGTGGGAATTCCGATAATCAACCTCTGGATTACAAAAT
TTGTGAAAGATTGACTGGTATTCTTAACTATGTTGCTCCTTTTACGCTATGTGGAT
ACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTCCCTATT
GCCACGGCGGAACTCATCGCCGCCTGCCTTGCCCGCTGCTGGACAGGGGCTCGG
CTGTTGGGCACTGACAATTCCGTGGTGTTGTCGGGGAAGCTGACGTCCTTTCCAT
GGCTGCTCGCCTGTGTTGCCACCTGGATTCTGCGCGGGACGTCCTTCTGCTACGT
CCCTTCGGCCCTCAATCCAGCGGACCTTCCTTCCCGCGGCCTGCTGCCGGCTCTG
CGGCCTCTTCCGCGTCTTCGCCTTCGCCCTCAGACGAGTCGGATCTCCCTTTGGGC
CGCCTCCCCGCATCGGGAATTCCTAGAGCTCGCTGATCAGCCTCGACTGTGCCTT
CTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTC
TGAGTAGGTGTCATTCTATTCTGGGGGGTGGGGTGGGGCAGGACAGCAAGGGGG
AGGATTGGGAAGAGAATAGCAGGCATGCTGGGGAGGGCCGCAGGAACCCCTAG
TGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCG
AGCGCGCAGCTGCCTGCAGGGGCGCCTGATGCGGTATTTTCTCCTTACGCATCTG
TGCGGTATTTCACACCGCATACGTCAAAGCAACCATAGTACGCGCCCTGTAGCGG
CGCATTAAGCGCGGCGGGGGTGGTGGTTACGCGCAGCGTGACCGCTACACTTGC
CAGCGCCTTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAG
TGCTTTACGGCACCTCGACCCCAAAAAACTTGATTTGGGTGATGGTTCACGTAGT
GGGCCATCGCCCTGATAGACGGTTTTTCGCCCTTTGACGTTGGAGTCCACGTTCTT
TAATAGTGGACTCTTGTTCCAAACTGGAACAACACTCAACTCTATCTCGGGCTAT
TCTTTTGATTTATAAGGGATTTTGCCGATTTCGGTCTATTGGTTAAAAAATGAGCT
GATTTAACAAAAATTTAACGCGAATTTTAACAAAATATTAACGTTTACAATTTTA
TGGTGCACTCTCAGTACAATCTGCTCTGATGCCGCATAGTTAAGCCAGCCCCGAC
ACCCGCCAACACCCGCTGACGCGCCCTGACGGGCTTGTCTGCTCCCGGCATCCGC
TTACAGACAAGCTGTGACCGTCTCCGGGAGCTGCATGTGTCAGAGGTTTTCACCG
TCATCACCGAAACGCGCGAGACGAAAGGGCCTCGTGATACGCCTATTTTTATAG
GTTAATGTCATGATAATAATGGTTTCTTAGACGTCAGGTGGCACTTTTCGGGGAA
ATGTGCGCGGAACCCCTATTTGTTTATTTTTCTAAATACATTCAAATATGTATCCG
CTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTT
CCTGTTTTTGCTCACCCAGAAACGCTGGTGAAAGTAAAAGATGCTGAAGATCAGT
TGGGTGCACGAGTGGGTTACATCGAACTGGATCTCAACAGCGGTAAGATCCTTG
AGAGTTTTCGCCCCGAAGAACGTTTTCCAATGATGAGCACTTTTAAAGTTCTGCT
ATGTGGCGCGGTATTATCCCGTATTGACGCCGGGCAAGAGCAACTCGGTCGCCG
CATACACTATTCTCAGAATGACTTGGTTGAGTACTCACCAGTCACAGAAAAGCAT
CTTACGGATGGCATGACAGTAAGAGAATTATGCAGTGCTGCCATAACCATGAGT
GATAACACTGCGGCCAACTTACTTCTGACAACGATCGGAGGACCGAAGGAGCTA
ACCGCTTTTTTGCACAACATGGGGGATCATGTAACTCGCCTTGATCGTTGGGAAC
CGGAGCTGAATGAAGCCATACCAAACGACGAGCGTGACACCACGATGCCTGTAG
CAATGGCAACAACGTTGCGCAAACTATTAACTGGCGAACTACTTACTCTAGCTTC
CCGGCAACAATTAATAGACTGGATGGAGGCGGATAAAGTTGCAGGACCACTTCT
GCGCTCGGCCCTTCCGGCTGGCTGGTTTATTGCTGATAAATCTGGAGCCGGTGAG
CGTGGAAGCCGCGGTATCATTGCAGCACTGGGGCCAGATGGTAAGCCCTCCCGT
ATCGTAGTTATCTACACGACGGGGAGTCAGGCAACTATGGATGAACGAAATAGA
CAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGAT
CTAGGTGAAGATCCTTTTTGATAATCTCATGACCAAAATCCCTTAACGTGAGTTTT
CGTTCCACTGAGCGTCAGACCCCGTAGAAAAGATCAAAGGATCTTCTTGAGATCC
TTTTTTTCTGCGCGTAATCTGCTGCTTGCAAACAAAAAAACCACCGCTACCAGCG
GTGGTTTGTTTGCCGGATCAAGAGCTACCAACTCTTTTTCCGAAGGTAACTGGCT
TCAGCAGAGCGCAGATACCAAATACTGTTCTTCTAGTGTAGCCGTAGTTAGGCCA
CCACTTCAAGAACTCTGTAGCACCGCCTACATACCTCGCTCTGCTAATCCTGTTA
CCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACAC
AGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTT
TTTACGGTTCCTGGCCTTTTGCTGGCCTTTTGCTCACATGT

### Example 7

An AAV vector delivering hrFGF18 without codon optimization under the regulation of a CAG promoter and containing a WPRE post-translational regulatory element.

### Example 8

The effects on cartilage anabolism of rhFGF18 protein and AAV2-delivered hFGF18 gene therapy were compared. Chondrogenic properties of AAV2-FGF18 were compared to full-length, fully-glycosylated rhFGF18 protein and E. coli expressed rhFGF18 protein analog in vitro relative to PBS and AAV2-GFP negative controls. Gene expression analysis was performed using RNA-seq on primary human chondrocytes from middle-aged adults. In vivo cartilage anabolism was evaluated by confirming long-term gene expression using AAV2-nLuc and measuring cartilage thickness in the tibial plateau and the white zone of the anterior hom of the medial meniscus in Sprague-Dawley rats following administration of increasing doses of AAV2-FGF18, rhFGF18 protein analog, and AAV2-GFP control.

AAV2-FGF18, AAV2-GFP, and AAV2-nLuc were manufactured using triple transfection in suspension cell culture of HEK293 cells, followed by downstream purification via clarification, ion exchange chromatography, and UF/DF, prior to controlled temperature freezing at −80C. Viral vector preparations were diluted to the target dose using 20 μL of room temperature 1×PBS solution, 30 minutes prior to injection. In vivo doses ranged between 2×10⁹ and 1×10¹¹ viral genomes per joint administered via a 20 uL intra-articular injection (Sprague Dawley rats) using a 30 G needle. In vitro doses ranged between MOI 10 and MOI 1000 vg/cell, viral vector preparations were diluted in 1×PBS, 1×PBS and 1% albumin solution, or 0.5% sucrose in USP WFI solution. Blinding and randomization best practices were applied to all experiments to the extent possible. Raw data was analyzed for normality using the Anderson-Darling normality test. Normally distributed data was analyzed using ANOVA, followed by Tukey's Post Hoc testing for multi-factorial experiments and Student's T-test for one factor, multi-level, or two-factor one level studies. Data are presented as mean±standard deviation. A significant difference was considered at a p-critical value (pcrit) of <0.05, except for RNA-Seq analysis where a pcrit of <0.01 was used to align with RNA-Seq best practices. All statistical analysis was performed using Minitab version 21.1.

### Results

AAV2-delivered hFGF18 represents a promising strategy for the restoration of hyaline articular cartilage by promoting anatomically relevant extracellular matrix production, chondrocyte proliferation, and increasing articular and meniscal cartilage thickness in vivo. FGF18 delivered as a protein or via an AAV2 vector demonstrates chondroanabolic activity by promoting chondrocyte proliferation and upregulation of hyaline cartilage associated genes including COL2A1 and HAS2, while downregulating fibrocartilage associated COL1A1. This activity translates to statistically significant cartilage thickness increases in vivo in the area of the tibial plateau and meniscal tip following a single intra-articular injection of the AAV2-FGF18 and a regimen of 6 bi-weekly injections of rhFGF18 protein relative to the AAV2-GFP control. Moreover, the single-injection AAV2-delivered hFGF18 offers a potential safety advantage over the multi-injection protein treatment as evidenced by reduced joint swelling over the treatment period.

To evaluate the cytocompatibility of the AAV2 vector, primary human chondrocytes and primary human synoviocytes were treated with increasing doses of AAV2 encoding a Green Fluorescent Protein (GFP) reporter transgene (FIG. 1) ranging from MOI of 1,000 to an MOI of 500,000. No dose-dependent cytotoxicity was observed in vitro for either chondrocytes or synoviocytes as evidenced by the lack of statistical significance between test groups (Dose) and test times (Time) (Two-Way ANOVA, p_{crit}=0.05), despite the high multiplicity of infection evaluated. Both synoviocytes and chondrocytes appeared highly permissive to AAV2-mediated transduction as evidenced by rapidly increasing GFP positive cell counts between 24 and 168 hours. Chondrocytes appeared to be more conducive to AAV2 transduction in vitro as an MOI of 50,000 for chondrocytes reached 97% GFP+ cells, while the same MOI only reached 58% GFP+ cells in synoviocytes culture.

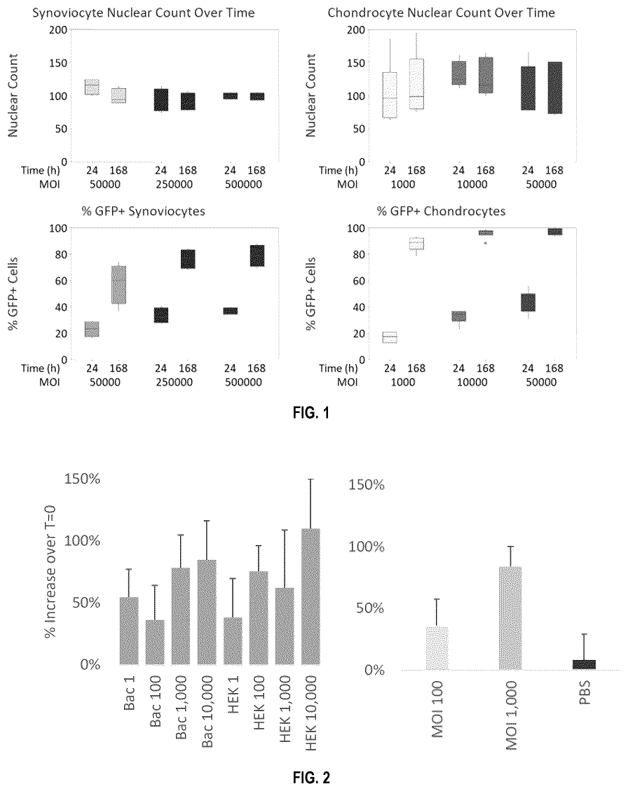

Since the rhFGF18 protein analog evaluated in clinical trials is bacterially expressed in E. coli (15), the ability of eukaryotically expressed hFGF18 to promote proliferation in a dose dependent manner relative to the bacterial rhFGF18 analog was tested. The E. coli expressed rhFGF18 protein analog (Bac) and HEK293 expressed rhFGF18 protein (HEK) demonstrated dose-dependent proliferation, which was not statistically different between the two test groups but demonstrated statistical significance for the dose factor (FIG. 2). A similar dose response curve was observed for the AAV2-FGF18 treated chondrocytes at a multiplicity of infection (MOI) of 100 and 1,000, with the MOI 1,000 dose reaching 84% increase in cell count relative to the pre-dosing timepoint (t=0), which was comparable to the 10,000 ng/mL dose of the bacterially expressed rhFGF18 analog (85%).

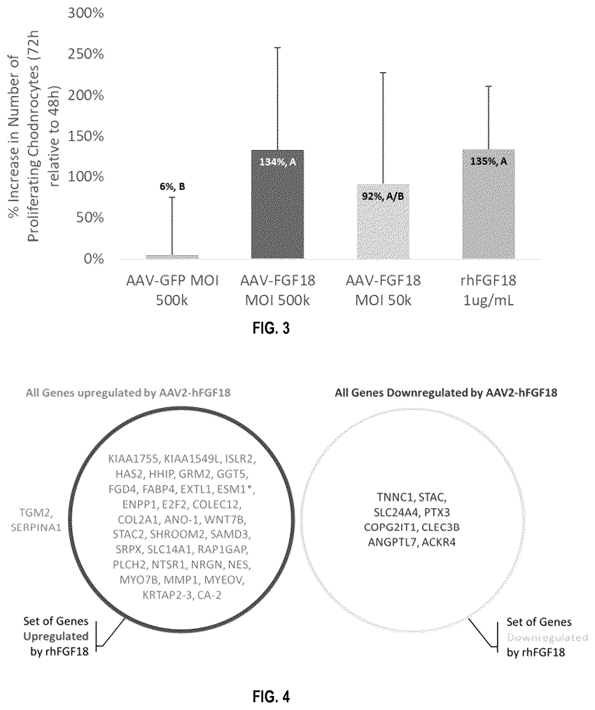

The effect of hFGF18 paracrine signaling from AAV2-FGF18 transduced synoviocytes was evaluated in transwell culture with primary human chondrocytes (FIG. 3). The number of proliferating chondrocytes increased by 92-135% between 48 and 72 h following exposure to AAV2-FGF18 and rhFGF18 protein treated synoviocytes, while an only 6% increase was observed for the AAV2-GFP negative control dosed at an MOI of 500,000.

In addition to the chondroproliferative effects of the hFGF18 protein and AAV2-FGF18 gene therapy, the gene expression profile of chondrocytes exposed to high doses of rhFGF18 protein (1,000 ng/mL) and sub-proliferative doses of the AAV2-FGF18 (MOI 10) was compared. The experimental design allowed for the identification of a holistic set of genes upregulated by the rhFGF18 protein for comparison with the most significantly upregulated subset from the AAV2-FGF18 gene therapy treated group. As a result, 94% or all but 2 genes (TGM2 and SERPINA1) upregulated in the AAV2-FGF18 treated chondrocytes at the 3× cutoff were also upregulated in the rhFGF18. Similarly, all genes (100%) downregulated by the AAV2-FGF18 gene therapy at the 3× cutoff, were also downregulated by the rhFGF18 treated chondrocytes (FIG. 4).

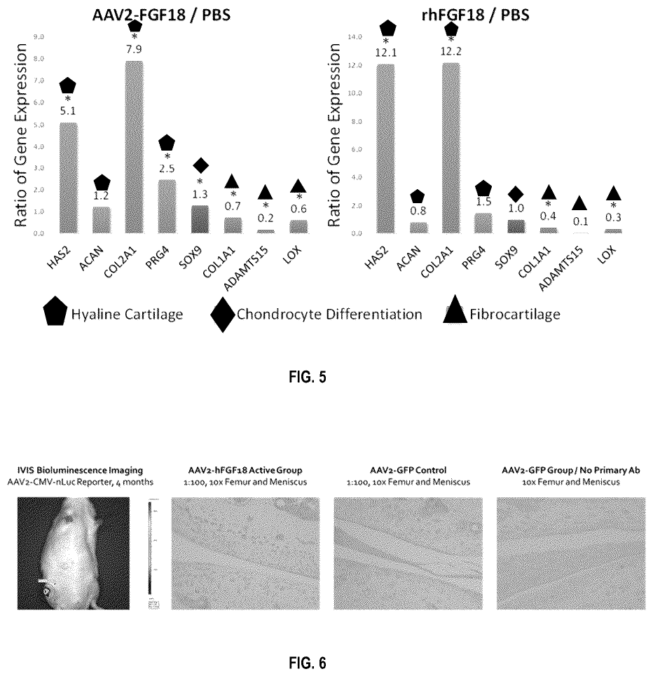

When analyzing genes specifically associated with the hyaline cartilage phenotype, including HAS2 (20), ACAN (21), COL2A1 (22), and PRG4 (23), AAV2-FGF18 treated chondrocytes upregulated all tested genes (with only ACAN not achieving statistical significance at a p_{crit}=0.01), while rhFGF18 protein treated chondrocytes only upregulated HAS2, COL2A1, and PRG4 (with PRG4 not achieving statistical significance at a p_{crit}=0.01) (FIG. 5). All fibrocartilage extracellular matrix associated genes (COL1A1 (24), ADAMTS15 (25), LOX (26)) were downregulated in the AAV2-FGF18 treated group and the rhFGF18 protein treated group (with ADAMTS15 not achieving statistical significance in the protein treated arm at a p_{crit}=0.01). Interestingly, the AAV2-FGF18 treated chondrocytes also upregulated the SOX9 chondrocyte differentiation marker (27), while a similar upregulation was not observed in the rhFGF18 protein treated group.

Gene expression was observed in vivo over a period of 4 months using nano-Luciferase reporter gene under the regulation of the CMV promoter, delivered via an AAV2 vector (FIG. 6). A strong bioluminescent signal was detected locally in the joint following intra-articular administration of the furimazine substrate. Signal intensity appeared to be most consistent when readings were taken 12 minutes following substrate injection using a 20 uL intra-articular injection volume. Additionally, hFGF18 expression was confirmed at the terminal study timepoint using 1:100 dilution of anti-hFGF18 antibody in the AAV2-FGF18 active group relative to AAV2-GFP control and AAV2-GFP with no-primary antibody negative control (all transgenes under the regulation of the same CMV promoter).

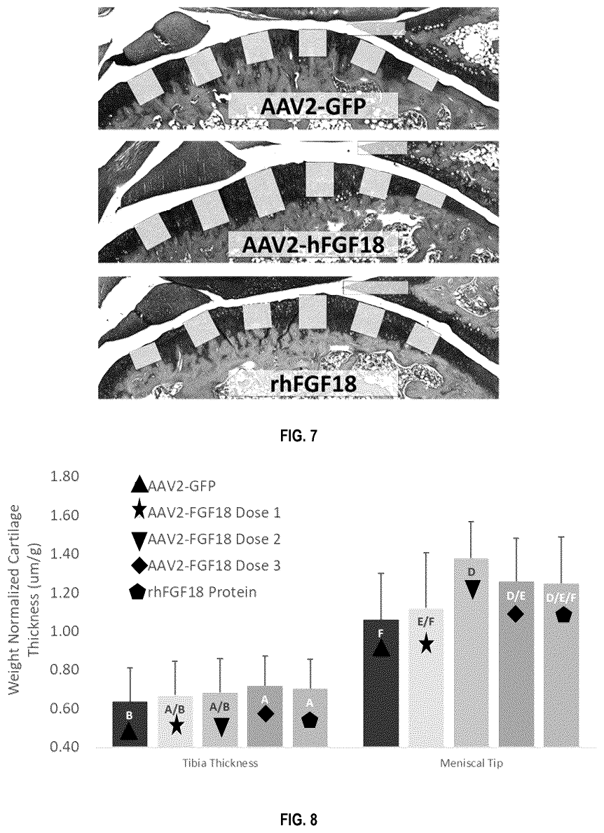

Analysis of saggital sections of knee joints demonstrated a qualitative increase in cartilage thickness in the AAV2-FGF18 and rhFGF18 treatment groups relative to the AAV2-GFP negative control over the tibial surface and at the meniscal tip (white zone of the anterior horn of the medial meniscus). No observable effects to the subchondral bone, marrow content or morphology, as well as the pars intermedia section of the meniscus were noted in any of the histological sections (FIG. 7). Finally, no signs of inflammation, degenerative processes within cartilage or the underlying bone, or abnormal growths were observed within the areas examined.

Quantitative, statistically significant increases in cartilage thickness were observed in the tibia and meniscal tip following normalization by animal weight (FIG. 8). The normalized tibial cartilage thickness for the AAV2-FGF18 gene therapy ranged between 0.72 0.15 um/g (average±standard deviation) and 0.67±0.18 um/g; the normalized tibial cartilage thickness for the rhFGF18 protein treatment arm was 0.70±0.15 um/g respectively.

The control AAV2-GFP dosed joints demonstrated an average cartilage thickness of 0.64±0.17 ug/g at the 2-month timepoint. A statistically significant difference in cartilage thickness was observed for the protein treatment arm and the highest gene therapy dose in the Tibia relative to the negative control, which is known for its lack of chondrogenic or chondrodegenerative properties (28-30) (One-Way ANOVA followed by Tukey's pairwise comparison post-hoc test, p_{crit}=0.05). Within the meniscus, the two top gene therapy doses attained statistically significant difference over the negative control, grouping together with the protein treatment, however, the protein did not achieve a statistically significant difference relative to the negative control using the Tukey's pairwise comparison post-hoc test. A dose response curve was observed in the Tibia, while Dose 2 AAV2-FGF18 appeared to perform best in the meniscal tip cartilage.

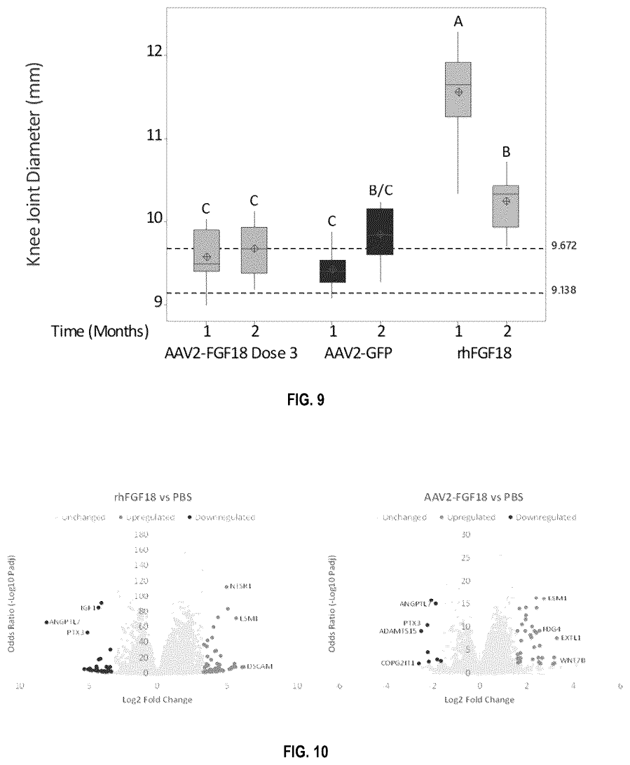

Finally, an increased joint swelling following repeat administration of FGF18 over the 2-month study duration was observed. The swelling was observed at both the 1- and 2-month timepoints, with partial resolution toward the end of the study (FIG. 9). At the 1 month timepoint the multi-injection rhFGF18 protein treated joints increased in diameter by 22.8% relative to no-injection baseline controls. Both the 1- and 2-month timepoints of the protein treatment arm were statistically different from all other test groups except the AAV2-GFP at 2 months (ANOVA, Tukey's pairwise comparison, p_{crit}=0.05).

### DISCUSSION

Osteoarthritis bears many of the hallmarks of a classical disease of aging, where age-related decreases in tissue cellularity as well as structurally important ECM-deposition result in a progressively degenerative phenotype that eventually requires surgical intervention (4, 31). While inflammatory cytokines have been hypothesized to possess both leading and contributory roles (32, 33), none of the anti-inflammatory therapies investigated to date have been able to demonstrate disease modification in controlled, randomized clinical trials. Repeat administrations of the chondroanabolic rhFGF18 protein analog, Sprifermin, has demonstrated the ability to increase cartilage thickness against a placebo control (15). The protein injection approach, however, is a multi-dose therapy requiring up to 12 injections per year in bilateral osteoarthritis treatment and may need to be sustained indefinitely to prevent reversal of cartilage gains (15). To overcome the delivery challenges associated with rhFGF18 protein therapy, a hFGF18 gene therapy, using an AAV2 delivery vector was developed.

The results demonstrate excellent transfection efficiency and cytocompatibility of the AAV2 vector with primary human synoviocytes and chondrocytes, the cell types that have been suggested as the primary targets for stable transgene expression in the joint (35). Chondrocytes appeared to be more conducive to AAV2-mediated transduction as evidenced by higher percentage of GFP positive cells at significantly lower doses. After 168 h in culture, >90% of chondrocytes were assessed as GFP+ by quantitative fluorescent microscopy at an MOI of 1,000, while only ˜80% GFP+ synoviocytes were observed over the same timeframe at an MOI of 500,000 AAV2-GFP per cell (FIG. 1). Despite these differences in transduction efficiency, both cells expressed significant amounts of GFP protein and did not demonstrate a statistically significant decline in cell count for any of the evaluated doses, confirming no dose-dependent cytotoxicity of the delivery vector.

In parallel, significantly lower doses of AAV2-FGF18 (MOI 100 and MOI 1,000) were able to induce chondroproliferative effects of a similar magnitude to that observed for protein doses previously evaluated in vitro to inform in vivo and subsequent clinical dose selection (36) (FIG. 2). Since this effect could be attributed to the previously suggested autocrine activity of the FGF18 protein (37) and transduction of synoviocytes may predominate in vivo despite their lower transduction potential in vitro, the paracrine effect was further evaluated using a trans-well co-culture assay. Synoviocytes transduced with AAV2-FGF18 cultured in transwell plates with primary human synoviocytes confirmed the ability of AAV2-FGF18 to mediate chondrocyte proliferation in a paracrine manner at a magnitude comparable to lug/mL of rhFGF18 protein. These results support the potential of AAV2-FGF18 gene therapy to promote chondroproliferative effects following intra-articular delivery, regardless of the precise biodistribution within the joint, so long as at least some of the resident cells proximal to the joint capsule are transduced.

Subsequently, the ability of AAV2-FGF18 gene therapy to promote the upregulation of hyaline cartilage associated genes, while downregulating fibrocartilage associated genes in culture, was assessed. Hyaline cartilage is the natural cartilage form of cartilage in articular joints, while the presence of fibrocartilage following surgical focal defect repair procedures (such as microfracture) was previously suggested to result in decreased durability of repair (38). Treatment of primary human chondrocytes with AAV2-FGF18 and rhFGF18 protein upregulated HAS2 and COL2A1 relative to the PBS baseline; however, only AAV2-FGF18 was able to achieve a statistically significant increase in PRG4. Since HAS2 is an essential hyaluronan synthesis component, specifically responsible for the production of high molecular mass hyaluronan, which is abundant in hyaline cartilage (20), while COL2A1 and PRG4 are secreted proteins and essential components of hyaline cartilage (22, 23), with structural and anti-adhesion-specific roles (39), the upregulation of these hyaline cartilage associated genes was further supportive of hFGF18's chondroanabolic activity.

Conversely, COL1A1 and LOX were downregulated in the protein and AAV2-FGF18 treatment groups, while only the AAV2-FGF18 treatment group was able to achieve a statistically significant reduction in ADAMTS15. COL1A1 is a fibrocartilage matrix component, which is upregulated in abnormal cartilage repair associated following microfracture (40). Similarly, Lysyl oxidase family cuproenzymes (LOX) were suggested to play a role in collagen crosslinking and LOX modulation has been observed to promote cartilage regeneration (26). Similarly, ADAMTS15, an important metalloproteinase responsible for catabolic activity (41), has been associated with pathologic osteoarthritis, and modulation of ADAMTS15 was suggested as a potentially viable approach to prevent progressive cartilage degeneration (42).

Interestingly, Sox9, a chondrocyte differentiation associated transcription factor, was only upregulated in the AAV2-FGF18 and not the rhFGF18 protein treatment group; however, the degree of upregulation was relatively small compared to baseline, only achieving a 1.3× multiple. A number of other similarly upregulated and downregulated genes was observed including ESM1, NTSR1, MMP1, and ANGPTL7, COPG2IT1, PTX3 respectively (FIG. 10), all which have previously been previously associated with skeletal development and maintenance (43-48). These findings further suggest similarity in activity between the rhFGF18 protein and AAV2-FGF18 gene therapy, regardless of the different vehicle of protein delivery. Conversely, the result of differential upregulation by AAV2-FGF18 and not the rhFGF18 protein of TGM2, which is a calcium-dependent acyltransferase with observed involvement in cartilage homeostasis (49) and SERPINA1, which has been previously associated with chondrogenesis and chondrocyte differentiation (50) require further investigation using pathway analysis and mechanistic studies to elucidate whether the differences can be attributed to the degree of hFGF18 accumulation in the cytoplasm, delivery, or pathways activated by the AAV2 capsid.

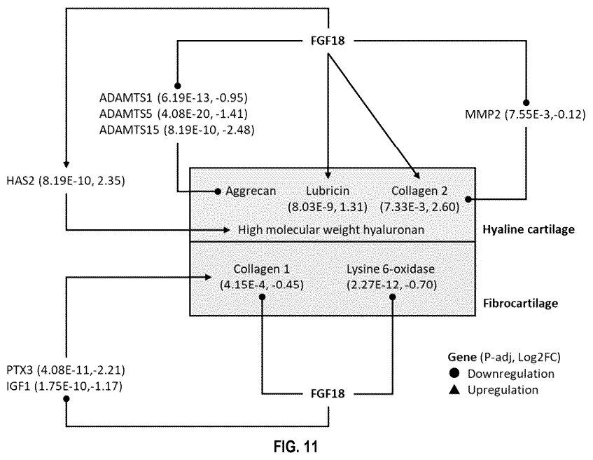

All in all, the results of gene expression analysis suggest that the mechanism of hFGF18 activity is comprised of two complementary anabolic components driving chondrocyte proliferation and hyaline cartilage extracellular matrix production (FIG. 11) with multiple direct (Lubricin/PRG4, Collagen 2/COL2A1) and indirect pathways (ADAMTS1, 5, 15, and MMP2) (51-53). In parallel, FGF18 appears to suppress expression of fibrocartilage associated genes via direct downregulation of COL1A1 and LOX, as well as indirect downregulation of genes that promote fibrosis including PTX3 and IGF1 (54, 55).

The safety of the AAV2 delivery vector was confirmed by administering doses up to 1×10¹¹ vg per joint via intra-articular injection. No abnormal growths or tumors in cartilage, meniscus, sub-chondral bone, or the proximal bone marrow were observed over the study duration. Furthermore, neither the AAV2-GFP control nor the AAV2-FGF18 gene therapy treatment arm demonstrated any clearly observable chondro- or osteo-degenerative processes, inflammatory infiltrates, or other qualitative attributes of tissue degradation or cellular inflammation, which was in line with previous studies using viral-vector delivered gene therapy (18, 34, 37).

Statistically significant increases in articular and meniscal cartilage thickness were observed following the delivery of the hFGF18 transgene and rhFGF18 protein analog (37). Both the rhFGF18 protein injection treatment and AAV-hFGF18 treatment arm demonstrated statistically significant increases in tibial cartilage thickness over the AAV2-GFP negative control reaching increases of 9.4% and 12.5% respectively. The highest dose of the AAV2-FGF18 group and the protein treatment group achieved statistical significance over the negative control in the Tibia. While previous studies, using smaller numbers of animals and without the use of weight normalization did not observe statistically significant increases in cartilage thickness of healthy rodent joints dosed using rhFGF18 protein, the studies were limited by their sample size and lack weight normalization, which was previously demonstrated to be associated with cartilage thickness (16). Moreover, early studies using adenoviral vector delivered hFGF18 observed chondrocyte proliferation, upregulation of extracellular matrix production, and locally increased tissue thickness attributed to increased cartilage accumulation (37). While the overall increase in cartilage thickness is relatively small, comprising approximately 28-37 um of additional cartilage (relative to the control baseline of 298 um), thinning of hyaline cartilage has been demonstrated to occur as a function of age and progression of osteoarthritis (8-10), as such, these increases may be sufficient to delay the onset of symptomatic osteoarthritis or slow the age-related erosion of cartilage over longer timeframes. Interestingly, the increase in cartilage thickness was most pronounced at the meniscal tip, where administration of the middle dose of AAV2-FGF18 resulted in a 30% increase in the white cartilaginous zone relative to the AAV2-GFP control. The protein treatment group increased the thickness of the meniscal tip by 18%; however, statistical significance was not achieved, likely due to the sample size and magnitude of effect. This increase in cartilage correlated with the meniscal section, which is known to be comprised of a more hyaline-cartilage phenotype with increased concentration of Type II collagen (56, 57). While chondroregenerative treatments focused on articular cartilage are currently under late-stage clinical evaluation, the application of said treatments to repair of meniscal tissues has not been previously reported. At the same time, no joint swelling following single injections of AAV2-GFP or AAV2-FGF18 was observed, whereas the protein treatment arm demonstrated significantly increased joint diameters relative to the uninjected baseline controls peaking at the 1-month timepoint and partially resolving by the second month. While it is unclear whether swelling is a phenomenon that will translate to the human joint, and the rhFGF18 protein injection treatment appears safe in placebo controlled randomized human trials (15), further evaluation of the multi-dose associated swelling effect with a focus on joint-to-dosing-needle aspect ratio and injection frequency is warranted.

In conclusion, hFGF18 gene therapy demonstrates a number of mechanistic parallels with rhFGF18 protein analog activity, which is currently being developed for the treatment of osteoarthritis and has been demonstrated to promote increases in articular cartilage thickness in vivo (15, 16). The ability to increase chondrocyte proliferation, upregulate several hyaline cartilage extracellular matrix related genes while downregulating fibrocartilage associated genes, and promote the increase of cartilage thickness in rat knee joints, supports the potential advancement of the hFGF18 gene therapy into disease model efficacy testing in rodent models of osteoarthritis and focal cartilage lesion repair. Finally, the observed effects of rhFGF18 protein and hFGF18 gene therapy on the meniscal tip support hFGF18's potential to promote regenerative repair of meniscal tears.

### REFERENCES

1. Farage M A, Miller K W, Elsner P, Maibach H I. Characteristics of the Aging Skin. Adv Wound Care (New Rochelle). 2013; 2(1):5-10. doi:10.1089/wound.2011.0356
2. SugiyamaY, Naito K, Goto K, et al. Effect of aging on the tendon structure and tendon-associated gene expression in mouse foot flexor tendon. Biomed Rep. 2019; 10(4):238-244. doi:10.3892/br.2019.1200
3. Ramasamy T S, Yee Y M, Khan I M. Chondrocyte Aging: The Molecular Determinants and Therapeutic Opportunities. Front Cell Dev Biol. 2021; 9:625497. Published 2021 July 14. doi:10.3389/fcell.2021.625497
4. Lotz M, Loeser R F. Effects of aging on articular cartilage homeostasis. Bone. 2012; 51(2):241-248. doi:10.1016/j.bone.2012.03.023
5. Mirahmadi F, Koolstra J H, Fazaeli S, et al. Aging does not change the compressive stiffness of mandibular condylar cartilage in horses. Osteoarthritis Cartilage. 2018; 26(12):1744-1752. doi:10.1016/j.joca.2018.08.007
6. Martin J A, Buckwalter J A. Roles of articular cartilage aging and chondrocyte senescence in the pathogenesis of osteoarthritis. Iowa Orthop J. 2001; 21:1-7
7. Palazzo C, Nguyen C, Lefevre-Colau M M, Rannou F, Poiraudeau S. Risk factors and burden of osteoarthritis. Ann Phys Rehabil Med. 2016; 59(3):134-138. doi:10.1016/j.rehab.2016.01.006
8. Khalil, N. F. W., El-sherif, S., El Hamid, M. M. A. et al. Role of global femoral cartilage in assessing severity of primary knee osteoarthritis. Egypt Rheumatol Rehabil 49, 16 (2022). doi.org/10.1186/s43166-022-00115-3
9. Erhart-Hledik J C, Favre J, Andriacchi T P. New insight in the relationship between regional patterns of knee cartilage thickness, osteoarthritis disease severity, and gait mechanics. J Biomech. 2015; 48(14):3868-3875. doi:10.1016/j.jbiomech.2015.09.033
10. Hadjab I, Sim S, Karhula S S, et al. Electromechanical properties of human osteoarthritic and asymptomatic articular cartilage are sensitive and early detectors of degeneration. Osteoarthritis Cartilage. 2018; 26(3):405-413. doi:10.1016/j.joca.2017.12.002
11. Perry T A, Wang X, Nevitt M, Abdelshaheed C, Arden N, Hunter D J. Association between current medication use and progression of radiographic knee osteoarthritis: data from the osteoarthritis initiative. Rheumatology (Oxford). 2021; 60(10):4624-4632. doi:10.1093/rheumatology/keab059
12. McAlindon T E, LaValley M P, Harvey W F, et al. Effect of Intra-articular Triamcinolone vs Saline on Knee Cartilage Volume and Pain in Patients With Knee Osteoarthritis: A Randomized Clinical Trial. JAMA. 2017; 317(19):1967-1975. doi:10.1001/jama.2017.5283
13. Zeng C, Lane N E, Hunter D J, et al. Intra-articular corticosteroids and the risk of knee osteoarthritis progression: results from the Osteoarthritis Initiative. Osteoarthritis Cartilage. 2019; 27(6):855-862. doi:10.1016/j.joca.2019.01.007
14. Nielsen F K, Egund N, Jorgensen A, Jurik A G. Risk factors for joint replacement in knee osteoarthritis; a 15-year follow-up study. BMC Musculoskelet Disord. 2017; 18(1):510. Published 2017 Dec. 4. doi:10.1186/sl2891-017-1871-z
15. Eckstein F, Hochberg M C, Guehring H, et al. Long-term structural and symptomatic effects of intra-articular sprifermin in patients with knee osteoarthritis: 5-year results from the FORWARD study [published online ahead of print, 2021 May 7]. Ann Rheum Dis. 2021; 80(8):1062-1069. doi:10.1136/annrheumdis-2020-219181
16. Moore E E, Bendele A M, Thompson D L, et al. Fibroblast growth factor-18 stimulates chondrogenesis and cartilage repair in a rat model of injury-induced osteoarthritis.

Osteoarthritis Cartilage. 2005; 13(7):623-631. doi:10.1016/j.joca.2005.03.003
17. Barr L, Getgood A, Guehring H, Rushton N, Henson F M. The effect of recombinant human fibroblast growth factor-18 on articular cartilage following single impact load.

J Orthop Res. 2014; 32(7):923-927. doi:10.1002/jor.22622
18. Payne K A, Lee H H, Haleem A M, et al. Single intra-articular injection of adeno-associated virus results in stable and controllable in vivo transgene expression in normal rat knees. Osteoarthritis Cartilage. 2011; 19(8):1058-1065. doi: 10.1016/j.joca.2011.04.009
19. Chen Q, Luo H, Zhou C, et al. Comparative intra-articular gene transfer of seven adeno-associated virus serotypes reveals that AAV2 mediates the most efficient transduction to mouse arthritic chondrocytes. PLoS One. 2020; 15 (12):e0243359. Published 2020 Dec. 15. doi:10.1371/journal.pone.0243359
20. Hiscock D R, Caterson B, Flannery C R. Expression of hyaluronan synthases in articular cartilage. Osteoarthritis Cartilage. 2000; 8(2):120-126. doi:10.1053/joca.1999.0280
21. Hascall, V. C., Heinegard, D. K., Wight, T. N. (1991). Proteoglycans. In: Hay, E. D. (eds) Cell Biology of Extracellular Matrix. Springer, Boston, M A. https://doi.org/10.1007/978-1-4615-3770-O_6
22. Antipova 0, Orgel J P. In situ D-periodic molecular structure of type I I collagen. J Biol Chem. 2010; 285(10):7087-7096. doi:10.1074/jbc.M109.060400
23. Satake, T., Komura, S., Aoki, H. et al. Induction of iPSC-derived Prg4-positive cells with characteristics of superficial zone chondrocytes and fibroblast-like synovial cells.

BMC Mol and Cell Biol 23, 30 (2022). https://doi.org/10.1186/s12860-022-00431-8
24. von der Mark K, Gauss V, von der Mark H, Muller P. Relationship between cell shape and type of collagen synthesised as chondrocytes lose their cartilage phenotype in culture. Nature. 1977; 267(5611):531-532. doi:10.1038/267531a0
25. Dancevic C M, McCulloch D R, Ward A C. The ADAMTS hyalectanase family: biological insights from diverse species. Biochem J. 2016; 473(14):2011-2022. doi: 10.1042/BCJ20160148
26. Lin W, Xu L, Li G. Molecular Insights Into Lysyl Oxidases in Cartilage Regeneration and Rejuvenation [published correction appears in Front Bioeng Biotechnol. 2020 Oct. 26; 8:598323]. Front Bioeng Biotechnol. 2020; 8:359. Published 2020 Apr. 30. doi: 10.3389/fbioe.2020.00359
27. Matsushita M, Kitoh H, Kaneko H, et al. A novel SOX9 H169Q mutation in a family with overlapping phenotype of mild campomelic dysplasia and small patella syndrome.

Am J Med Genet A. 2013; 161A(10):2528-2534. doi:10.1002/ajmg.a.36134
28. Yoon D S, Lee K M, Cho S, et al. Cellular and Tissue Selectivity of AAV Serotypes for Gene Delivery to Chondrocytes and Cartilage. Int J Med Sci. 2021; 18(15):3353-3360. Published 2021 Jul. 25. doi:10.7150/ijms.56760
29. Pagnotto M R, Wang Z, Karpie J C, Ferretti M, Xiao X, Chu C R. Adeno-associated viral gene transfer of transforming growth factor-beta1 to human mesenchymal stem cells improves cartilage repair. Gene Ther. 2007; 14(10):804-813. doi:10.1038/sj.gt.3302938
30. Chen Q, Luo H, Zhou C, et al. Comparative intra-articular gene transfer of seven adeno-associated virus serotypes reveals that AAV2 mediates the most efficient transduction to mouse arthritic chondrocytes. PLoS One. 2020; 15 (12):e0243359. Published 2020 Dec. 15. doi:10.1371/journal.pone.0243359
31. Carames B, Taniguchi N, Otsuki S, Blanco F J, Lotz M. Autophagy is a protective mechanism in normal cartilage, and its aging-related loss is linked with cell death and osteoarthritis. Arthritis Rheum. 2010; 62(3):791-801.
32. Kapoor M, Martel-Pelletier J, Lajeunesse D, Pelletier J P, Fahmi H. Role of proinflammatory cytokines in the pathophysiology of osteoarthritis. Nat Rev Rheumatol. 2011; 7(1):33-42. doi:10.1038/nrrheum.2010.196
33. Wojdasiewicz P, Poniatowski L A, Szukiewicz D. The role of inflammatory and anti-inflammatory cytokines in the pathogenesis of osteoarthritis. Mediators Inflamm. 2014; 2014:561459. doi:10.1155/2014/561459
34. Goodrich L R, Choi V W, Carbone B A, McIlwraith C W, Samulski R J. Ex vivo serotype-specific transduction of equine joint tissue by self-complementary adeno-associated viral vectors. Hum Gene Ther. 2009; 20(12):1697-1702. doi:10.1089/hum.2009.030
35. Evans C H, Ghivizzani S C, Robbins P D. Gene Delivery to Joints by Intra-Articular Injection. Hum Gene Ther. 2018; 29(1):2-14. doi:10.1089/hum.2017.181
36. Gigout A, Guehring H, Froemel D, et al. Sprifermin (rhFGF18) enables proliferation of chondrocytes producing a hyaline cartilage matrix. Osteoarthritis Cartilage. 2017; 25(11):1858-1867. doi:10.1016/j.joca.2017.08.004
37. Ellsworth J L, Berry J, Bukowski T, et al. Fibroblast growth factor-18 is a trophic factor for mature chondrocytes and their progenitors [published correction appears in Osteoarthritis Cartilage 2002 October; 10(10):826]. Osteoarthritis Cartilage. 2002; 10(4):308-320. doi:10.1053/joca.2002.0514
38. Gudas R, Gudaite A, Mickevicius T, et al. Comparison of osteochondral autologous transplantation, microfracture, or debridement techniques in articular cartilage lesions associated with anterior cruciate ligament injury: a prospective study with a 3-year follow-up. Arthroscopy. 2013; 29(1):89-97. doi:10.1016/j.arthro.2012.06.009
39. Rhee D K, Marcelino J, Baker M, et al. The secreted glycoprotein lubricin protects cartilage surfaces and inhibits synovial cell overgrowth. J Clin Invest. 2005; 115(3):622-631. doi: 10. 1 172/JC122263
40. Iseki T, Rothrauff B B, Kihara S, et al. Dynamic Compressive Loading Improves Cartilage Repair in an In Vitro Model of Microfracture: Comparison of 2 Mechanical Loading Regimens on Simulated Microfracture Based on Fibrin Gel Scaffolds Encapsulating Connective Tissue Progenitor Cells. Am J Sports Med. 2019; 47(9):2188-2199. doi:10.1177/0363546519855645
41. Porter S, Clark I M, Kevorkian L, Edwards D R. The ADAMTS metalloproteinases. Biochem J. 2005; 386 (Pt 1):15-27. doi:10.1042/BJ20040424
42. Yang C Y, Chanalaris A, Troeberg L. ADAMTS and ADAM metalloproteinases in osteoarthritis—looking beyond the ‘usual suspects’. Osteoarthritis Cartilage. 2017; 25(7):1000-1009. doi:10.1016/j.joca.2017.02.791
43. Sivaraj K K, Jeong H W, Dharmalingam B, et al. Regional specialization and fate specification of bone stromal cells in skeletal development. Cell Rep. 2021; 36(2):109352. doi:10.1016/j.celrep.2021.109352
44. Lian Q, Lye E, Suan Yeo K, et al. Derivation of clinically compliant MSCs from CD105+, CD24− differentiated human ESCs. Stem Cells. 2007; 25(2):425-436. doi: 10.1634/stemcells.2006-0420
45. Sasaki K, Takagi M, Konttinen Y T, et al. Upregulation of matrix metalloproteinase (MMP)-1 and its activator MMP-3 of human osteoblast by uniaxial cyclic stimulation. J Biomed Mater Res B Appl Biomater. 2007; 80(2):491-498. doi:10.1002/jbm.b.30622
46. Bratus-Neuenschwander A, Castro-Giner F, Frank-Bertoncelj M, et al. Pain-Associated Transcriptome Changes in Synovium of Knee Osteoarthritis Patients. Genes (Basel). 2018; 9(7):338. Published 2018 Jul. 4. doi:10.3390/genes9070338
47. Zhang Y, Fukui N, Yahata M, et al. Identification of DNA methylation changes associated with disease progression in subchondral bone with site-matched cartilage in knee osteoarthritis. Sci Rep. 2016; 6:34460. Published 2016 Sep. 30. doi:10.1038/srep34460.
48. Visconti V V, Greggi C, Fittipaldi S, et al. The long pentraxin PTX3: a novel serum marker to improve the prediction of osteoporosis and osteoarthritis bone-related phenotypes [published correction appears in J Orthop Surg Res. 2021 May 21; 16(1):331]. J Orthop Surg Res. 2021; 16(1):288. Published 2021 Apr. 30. doi:10.1186/s13018-021-02440-3
49. Adamczyk M. Transglutaminase 2 in cartilage homoeostasis: novel links with inflammatory osteoarthritis. Amino Acids. 2017; 49(3):625-633. doi: 10.1007/s00726-016-2305-1
50. Boeuf S, Steck E, Pelttari K, et al. Subtractive gene expression profiling of articular cartilage and mesenchymal stem cells: serpins as cartilage-relevant differentiation markers. Osteoarthritis Cartilage. 2008; 16(1):48-60. doi:10.1016/j.joca.2007.05.008
51. Glasson S S, Askew R, Sheppard B, et al. Deletion of active ADAMTS5 prevents cartilage degradation in a murine model of osteoarthritis [published correction appears in Nature. 2007 Mar. 1; 446(7131):102]. Nature. 2005; 434(7033):644-648. doi:10.1038/nature03369
52. Page-McCaw A, Ewald A J, Werb Z. Matrix metalloproteinases and the regulation of tissue remodelling. Nat Rev Mol Cell Biol. 2007; 8(3):221-233. doi:10.1038/nrm2125
53. Sternlicht M D, Werb Z. How matrix metalloproteinases regulate cell behavior. Annu Rev Cell Dev Biol. 2001; 17:463-516. doi:10.1146/annurev.cellbio.17.1.463
54. Greevid D, Sironi M, Valentino S, et al. The Long Pentraxin 3 Plays a Role in Bone Turnover and Repair. Front Immunol. 2018; 9:417. Published 2018 Mar. 5. doi: 10.3389/fimmu.2018.00417
55. Blackstock C D, Higashi Y, Sukhanov S, et al. Insulin-like growth factor-1 increases synthesis of collagen type I via induction of the mRNA-binding protein LARP6 expression and binding to the 5′ stem-loop of COL1a1 and COL1a2 mRNA. J Biol Chem. 2014; 289(11):7264-7274. doi:10.1074/jbc.M113.518951
56. Hanifi A, McCarthy H, Roberts S, Pleshko N. Fourier transform infrared imaging and infrared fiber optic probe spectroscopy identify collagen type in connective tissues. PLoS One. 2013; 8 (5):e64822. Published 2013 May 22. doi:10.1371/joumal.pone.0064822
57. Sanchez-Adams J, Wilusz R E, Guilak F. Atomic force microscopy reveals regional variations in the micromechanical properties of the pericellular and extracellular matrices of the meniscus. J Orthop Res. 2013; 31(8):1218-1225. doi:10.1002/jor.22362

### Example 9

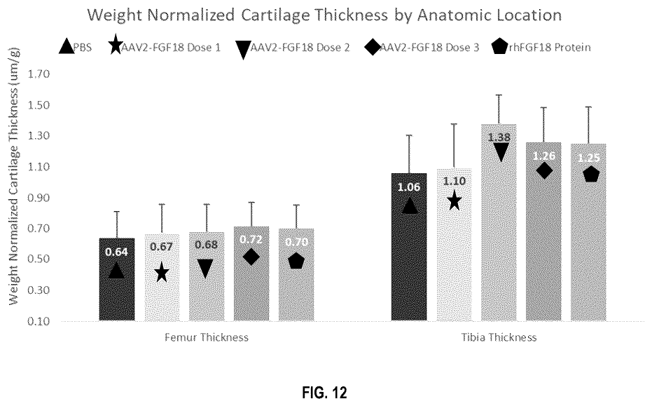

Three doses of a gene therapy treatment using a CMV promoter in the rat were tested (FIG. 12). Doses that are able to increase cartilage thickness relative to the negative control (PBS) include doses at 2×10⁹, 1×10¹⁰ and 1×10¹¹ vg/joint. In addition, the genetic constructs were found to outperform administered protein in the repair of the meniscus at doses that include the foregoing. For the experiments performed, a dose of 1×10¹⁰ vg/joint showed the highest effect in the meniscus, and a dose at 1×10¹¹ vg/joint in the tibia (articular cartilage).

Any one of the genetic constructs or compositions provided herein, can be in an amount or be administered in an amount such as to be delivered at any one of the doses provided herein. Accordingly, any one of the methods provided herein can comprise administering the genetic constructs or compositions provided herein such that any one of the doses provided herein are provided to a subject. The doses may be determined based on doses administered to a similar test subject, such as one similar with respect to any one or more of the following: species, age, weight, gender, and disease state. In another embodiment, the doses may be determined based on doses administered to a dissimilar test subject, such as dissimilar with respect to any one or more of the following: species, age, weight, gender, and disease state dissimilar, but adjusted. The adjustment can be based on average surface area of the joint, average joint volume, average volume articular cartilage between the subject (or what is expected for the subject) and test subject. Any one of the methods provided herein can include one or more steps for determining a dose and/or adjusting a dose of the foregoing.

For example, average joint volumes of rats and humans have been described:

| | | | |
|---|---|---|---|
| Rat | 30 | uL | pubmed.ncbi.nlm.nih.gov/31733365/ |
| joint | 130 | uL | pubmed.ncbi.nlm.nih.gov/23908112/ |
| volume | 100 | uL | pubmed.ncbi.nlm.nih.gov/24981310/ |
| Human | 0.5 | mL | tandfonline.com/doi/abs/10.3109/17453679209154758 |
| knee | 4 | mL | tandfonline.com/doi/abs/10.3109/17453679209154758 |
| joint | 1.5 | mL | ard.bmj.com/content/75/1/84 |
| | 8.5 | mL | ard.bmj.com/content/75/1/84 |
| Human | 500 | uL | tandfonline.com/doi/abs/10.3109/17453679209154758 |
| knee | 4000 | uL | tandfonline.com/doi/abs/10.3109/17453679209154758 |
| joint | 1500 | uL | ard.bmj.com/content/75/1/84 |
| | 8500 | uL | ard.bmj.com/content/75/1/84 |

As such, human dose ranges equivalent to the doses used in rats (using the lowest to highest ratio, highest to lowest ratio, and average joint volumes, respectively) can be determined. Exemplary values for illustration are provided below (per knee joint).

| Human Dose Absolute Max | 5.67E+11 | vg/joint |
|---|---|---|
| Human Dose Absolute Min | 1.00E+10 | vg/joint |
| Human Dose average | 8.37E+10 | vg/joint |
| Human Dose Absolute Max | 283 | Times increase over |
| Human Dose Absolute Min | 5 | rat dose |
| Human Dose average | 42 | |
| Human Dose Absolute Max | 2.83E+13 | vg/joint |
| Human Dose Absolute Min | 5.00E+11 | vg/joint |
| Human Dose average | 4.18E+12 | vg/joint |
| Human Dose Absolute Max | 283 | Times increase over |
| Human Dose Absolute Min | 5 | rat dose |
| Human Dose average | 42 | |

Similarly, doses for other species (low and high) may also be determined. Exemplary values for illustration are also provided.

| | | | |
|---|---|---|---|
| Dog | 0.5 | mL | pubmed.ncbi.nlm.nih.gov/ |
| | | | 27720280/ |
| | 1 | mL | pubmed.ncbi.nlm.nih.gov/ |
| | | | 27720280/ |
| Dog Dose Absolute Max | 3.33E+12 | vg/joint | |
| Dog Dose Absolute Min | 5.00E+11 | vg/joint | |
| Dog Dose average | 8.65E+11 | vg/joint | |
| Horse | 1.5 | mL | pubmed.ncbi.nlm.nih.gov/ |
| | | | 19544397/ |
| | 25 | mL | pubmed.ncbi.nlm.nih.gov/ |
| | | | 33861772/ |
| Horse Dose Absolute Max | 8.33E+13 | vg/joint | |
| Horse Dose Absolute Min | 1.50E+12 | vg/joint | |
| Horse Dose average | 1.53E+13 | vg/joint | |
| Dog Dose Absolute Max | 6.67E+10 | vg/joint | |
| Dog Dose Absolute Min | 1.00E+10 | vg/joint | |
| Dog Dose average | 1.73E+10 | vg/joint | |
| Horse Dose Absolute Max | 1.67E+12 | vg/joint | |
| Horse Dose Absolute Min | 3.00E+10 | vg/joint | |
| Horse Dose average | 3.06E+11 | vg/joint | |

Generally, higher doses may be used for more advanced disease, such as KL3 and KL4 osteoarthritis, while lower doses may be used in general for less advanced disease, such as KL1 and KL2 osteoarthritis, or as disease prevention. Any one of the methods provided herein may be used for the treatment of advanced disease or advanced cartilage loss, preferably at higher doses, such as any one of the higher doses as provided herein or as calculated or could be calculated herein. Any one of the methods provided herein may be used for the treatment of less advanced disease or for disease prevention, preferably at lower doses, such as any one of the lower doses as provided herein or as calculated or could be calculated herein. Any one of the methods provided herein, can include one or more steps for determining or adjusting a dose accordingly. Such determined or adjusted doses are also provided herein, and the doses of any one of the methods or compositions provided herein can be such doses.

In addition, a statistically significant correlation between the animal weight and the cartilage thickness was observed. The larger the animal weight, the smaller the cartilage at the meniscal tip, so dosing may also be determined by weight in any one of the methods provided herein. As an example:

Analysis of Variance

| Source | DF | Adj SS | Adj MS | F-Value | P-Value |
|---|---|---|---|---|---|
| Animal Weight | 1 | 30159 | 30159 | 4.19 | 0.043 |
| Test Arm | 4 | 164873 | 26218 | 3.64 | 0.008 |
| Error | 117 | 842253 | 7199 | | |
| Lack-of-fit | 24 | 331691 | 13820 | 2.52 | 0.001 |
| Pure Error | 93 | 510561 | 5490 | | |
| Total | 122 | 986233 | | | |

Regression Equation
Test Arm

| | |
|---|---|
| AAV2-FGF18 Dose 1 | Meniscal Tip = 297-0.624 Animal Weight |
| AAV2-FGF18 Dose 2 | Meniscal Tip = 354-0.624 Animal Weight |
| AAV2-FGF18 Dose 3 | Meniscal Tip = 283-0.624 Animal Weight |
| AAV2-GPP Dose 2 | Meniscal Tip = 318-0.624 Animal Weight |
| rhFGF18 protein | Meniscal Tip = 273-0.624 Animal Weight |

As an example, for humans, a higher dose range may be as follows:

| | | |
|---|---|---|
| Human Dose Absolute Max | 3.11E+11 | vg/joint/kg |
| Human Dose Absolute Min | 5.49E+09 | vg/joint/kg |
| Human Dose average | 4.60E+10 | vg/joint/kg |

As another example, for humans, a lower dose range may be as follows:

| | | |
|---|---|---|
| Human Dose Absolute Max | 6.23E+09 | vg/joint/kg |
| Human Dose Absolute Min | 1.10E+08 | vg/joint/kg |
| Human Dose average | 9.19E+08 | vg/joint/kg |

Correlate doses to the above may also be determined (such as for horses, dogs or cats) according to the principles provided herein, and such doses are also provided herein. Any one of the methods provided herein, can include one or more steps for determining or adjusting a dose accordingly. Such determined or adjusted doses are also provided herein, and the doses of any one of the methods or compositions provided herein can be such doses.

Finally, doses may be adjusted based on the promoter used. The CMV promoter is an example, and doses based on such a promoter may be determined. When using a different promoter, a dose may be determined and/or adjusted based on relative dose ratios dependent on the type of promoter (e.g., journals.plos.org/plosone/article?id=10.1371/joumal.pone.0010611 and nature.com/articles/gt20093).

Example ratios are provided below.

| Promoter | FITC-A | Relative Dose Ratio |
|---|---|---|
| CMV | 10500 | 1.00 |
| CAG | 17500 | 0.60 |
| SV40 | 9000 | 1.17 |
| TRE | 12000 | 0.88 |
| EF1a | 17000 | 0.62 |
| PGK | 5000 | 2.10 |
| UBC | 2500 | 4.20 |
| COL2A | 5000 | 2.10 |
| PRG4 | 2000 | 5.25 |
| ACAN | 2000 | 5.25 |
| HAS2 | 2500 | 4.20 |

Thus, any one of the doses provided herein may be adjusted based on the type of promoter of the genetic construct. Any one of the methods provided herein, can include one or more steps for determining or adjusting a dose accordingly. Such determined or adjusted doses are also provided herein, and the doses of any one of the methods or compositions provided herein can be such doses.

As another example, exemplary vectors may include a post-translational regulatory element (e.g., a WPRE regulatory element, such as an OPRE, WPREmut6, orWPREmutl). Such regulatory elements may reduce all dose ranges, such as by 25%, 20%, and 10%, respectively. Thus, doses may also be adjusted based on the regulatory elements of the genetic construct. Any one of the methods provided herein, can include one or more steps for determining or adjusting a dose accordingly. Such determined or adjusted doses are also provided herein, and the doses of any one of the methods or compositions provided herein can be such doses.

### Example 10

Provided are exemplary doses (average, high and low). Any one of these doses, or an equivalent as described herein, are applicable to any one of the compositions or methods provided herein. As can be appreciated, the doses provided herein also represent a dose range low to high. Any one of these dose ranges, or an equivalent as described herein, are applicable to any one of the compositions or methods provided herein.

| | | | | | |
|---|---|---|---|---|---|
| Human | 2.83E+13 | vg/joint | Human | 3.11E+11 | vg/joint/kg |
| Human | 5.00E+11 | vg/joint | Human | 5.49E+09 | vg/joint/kg |
| Human | 4.18E+12 | vg/joint | Human | 4.60E+10 | vg/joint/kg |

| | | |
|---|---|---|
| Dog | 3.33E+12 | vg/joint |
| Dose | | |
| Absolute | | |
| Max | | |
| Dog | 5.00E+11 | vg/joint |
| Dose | | |
| Absolute | | |
| Min | | |
| Dog | 8.65E+11 | vg/joint |
| Dose | | |
| average | | |
| Horse | 8.33E+13 | vg/joint |
| Dose | | |
| Absolute | | |
| Max | | |
| Horse | 1.50E+12 | vg/joint |
| Dose | | |
| Absolute | | |
| Min | | |
| Horse | 1.53E+13 | vg/joint |
| Dose | | |
| average | | |

1. A genetic construct comprising, a nucleic acid encoding a FGF-18 polypeptide, a promoter and, optionally, a regulatory element (such as a post-translational regulatory element) (e.g., is from the WPRE sequence family, encodes a PolyA signal, is an enhancer, is a translation termination sequence, is a sequence that promotes binding of one or more DNA binding proteins).

2. The genetic construct of claim 1, wherein the FGF-18 polypeptide is mammalian, such as human or non-human primate.

3. The genetic construct of claim 2, wherein the FGF-18 polypeptide is a human, dog, cat, cow, sheep, goat or horse FGF-18 polypeptide.

4. The genetic construct of claim 1, wherein the FGF-18 polypeptide is encoded by the sequence of AF075292, AB007422, AF211188, BT019570, BTo19571, CH471062, BC006245, AY358811, NM_003862.2 or a portion thereof.

5. The genetic construct of claim 1, wherein the FGF-18 polypeptide is encoded by at least a part of the sequence of gene ID 8817 from the HGNC:3674.

6. The genetic construct of claim 1, wherein the nucleic acid encodes another protein or portion thereof.

7. The genetic construct of claim 1, wherein the nucleic acid further encodes at least one auxiliary and/or regulatory sequence that facilitates expression.

8. The genetic construct of claim 1, wherein the nucleic acid further encodes at least one intron from another genetic sequence (e.g., human).

9. The genetic construct of claim 1, wherein the promoter is a/an CMV promoter, CMV promoter with an MVM1 intron, CAG promoter, EF1 alpha promoter, UBC promoter, CBh promoter, MSCV promoter, hPGK promoter, SFFV promoter, or SV40 promoter.

10. The genetic construct of claim 1, wherein the promoter is a constitutive promoter (e.g., mammalian).

11. The genetic construct of claim 1, wherein the promoter drives expression in at least one cell type at least transiently present within a joint or tissues surrounding a joint.

12. The genetic construct of claim 1, wherein the promoter is an inducible promoter.

13. The genetic construct of claim 12, wherein the inducible promoter up- or down-regulates expression in response to external or internal stimuli (e.g., inflammation, heat, light, stress, administration of steroids, tetracycline, antibiotics, rapamycin, ganciclovir, acyclovir) or is inducible by up- or down-regulated heat, ROS, NOS or cytokine release.

14. The genetic construct of claim 1, wherein the promoter is a circadian rhythm or cycling promoter, such as in response to cortisol levels, the menstrual cycle, the diurnal cycle, with the level of exercise, etc. (e.g., that changes its level of activity by at least 2%, 5% or 10% with some periodicity, such as from hours to months, including weekly).

15. The genetic construct of claim 1, wherein the promoter is a tissue-specific promoter.

16-20. (canceled)

21. A composition comprising the genetic construct of claim 1, wherein the nucleic acid is comprised in a delivery vector.

22-38. (canceled)

39. A method of administering the genetic constructs of claim 1, to a subject in need thereof.

40-77. (canceled)

78. A method comprising, determining or adjusting a dose based on using an average joint size (e.g., average joint surface area or average joint volume), average articular cartilage volume, weight, age and/or disease state of or representative of the subject relative to another subject, such as of a different species.

79-88. (canceled)

89. A composition comprising any of the genetic constructs provided herein at any one of the doses provided herein and a pharmaceutically acceptable carrier.

90. A genetic construct as described in any one of the Examples.


SEQUENCE LISTING (7 sequences)

SEQ ID NO: 1 - DNA, 2869 bp, synthetic construct
  source 1..2869: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcctcacaaagccgagtccagctggagccacctccagactctctggccagagcctcagagtggccaacagtccctggccagcggttgctctatccaggctaagggcacccactcccctggagattcctgaacctgggccaggaagagccgaactagacaagcgtctccaatccatcccgttttgtccccggggttgtctgcagtcccgttagtcccttccctggcctgttcgccccttagacctccacacaggtctccctctgtgtaggaattcaccagaccctgtcctagccacccgctcagttcccccttctacctaggccttttctagctagttggatgggggatgggttagggaggctgtggtccccgagactccaggtgggagctcacgggcaggtactccgcaaaggagctggaaggcaggtctggaaaactgtcccccagatttaggattctggacaacgtccatctgctcatgctttggtcccccacgcacctccccgcctcctaaattccccatccccacctttcttgctcctttctgttgcttcgtcctctctgccttgggtctaaaactccaggcttatgcctctgcaaacaaccccctcccttctaaccccagcagaactccgaggaaaggggcccgatgctcccccccttcccgcctgtggttagagggggcagtgtggcagtcccaagtgggggcgaccggaggctgtctcggtgccccgcccgatcaggccactgggcacattgggggcgggaagctgggctcacgaaaggggcgactggccttggcaggtgtgggctctggtccggcctgggcgggctccgggggcggggtctcaggttacagccccgcggggggctagggggcggcccgcggtttgggccggtttgccagcctttggagcgaccgggagcatataactggagcctctgccgggggaagacgcagagcgccgctgggctgccgggtctcctgcctcctcctgctcctagggcctcctgcatgagggagcggtagagaccaagtttgtacaaaaaagcaggctgccaccatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 2408 bp, synthetic construct
  source 1..2408: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgtacagcgctccaagcgcctgcacatgcctgtgtctgcacttcctgctgctgtgcttccaggtgcaggtgctggtggccgaggagaacgtggattttaggatccacgtggaaaaccagaccagagccagagatgatgtgagcaggaagcagctgcggctgtaccagctgtattcaaggaccagcggcaaacacatccaggtgctgggacggaggatctccgccagaggggaggacggtgacaagtacgcccagctgcttgtggagactgatacatttggctcccaggtgagaatcaagggcaaggagaccgagttttacctgtgcatgaacaggaaaggcaagctggtgggcaagccagacgggaccagcaaggagtgcgtgttcatcgagaaggtgctggagaacaactataccgccctgatgtccgccaagtactctggctggtacgtgggattcacaaagaagggaaggcctcgcaagggccctaagacccgggagaaccagcaggatgtccacttcatgaagagataccctaagggacagcccgaactgcagaagcccttcaagtacacaactgtgacaaagagaagcaggagaatcagaccaactcaccccgcctgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 2260 bp, synthetic construct
  source 1..2260: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 3368 bp, synthetic construct
  source 1..3368: mol_type = other DNA
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcaactttgtatagaaaagttgctcctgggctcccaaatgctggctccagtcaaggcttcagtcgtggtcctctgggagcatccaggctggggtggtttggcaaggggcttactagaccatcatgtgcagagagcagcagagccccttgtacaaaaaccaggacccctgggttccctgtggataggatgagctgccaaggagatgtcactcttggcaccctccaagaatcattacggccgggtgcagtggctcatgcttgaaatcccagcactttgggaggccgaggcgggtgaatcacctgaggtcaggagttcgagaccagcctggccaacatggtgaaatcctgtctctactaaaaatacaaaattagccaggcatggtggcatgtgcctgtaatcccagctactcgggaggctgaggcaggagaatcacttgaacccgggaggcggaggttgcagtcagccaagattgcgccactgtactccagcctgggcaacaagagtgaaactccatctcaaaaaaaaatacagaattagccaggcatggtggcgtgtgcctgtagtcccagctactcaagaggctgaggcaggagaatcacttgaacctgggaggcagaggttgcagtgagccgagatcgcaccattgcactcctgcctgggcgacagagcgagactccgtctcaaaaaaaaagaaccgcgagcactttgtttctcttgcttcttccacttcactcttcttcagccagggcagccaaaggcaggatgggtgcttcccggatccaccagagggggaaaggggttgggcgtgtatgggggcagaacttttccctagtgagagccttggtgaggtctgctggaggcacactggggacacaggcaagcatcaggtggtcccaacttcccacccccctcccataaacggcacttggctcagtctccctctgcccaccttgtcaccatggagctgtggcattcaagcatccaaatgccctgcttctgtcaggcctgccccgtcgggtgctttgtttacttgtcaggttgggcagatggtctcaagccctggttggtgggtgggtgagtgaggagacaggttagacaggggaaggtcccaggacatgtcttccctctctgggctgacctgaggcagtggaggctctcaggtgtgggatggggttttcaggctgggatgttctgtaccgtagaggataggccttcctattacttatcggagcactacgggagggcaggtccctccccagggtgtttaacactggaggctgcagggtcaggaggagaatcgtggggccaggagggcagaggcacactccatcttcgtgctcctcacaggccctgcctccctgcctgctaaggacacagggaagggggtccccacctcagtgcctgcctcccttccctgtgcctgtgtacctggcagtcacagccacctggcgtgtcccagaaaccaaccggctgacctcatctcctgcccggccccacctccattggctttggcttttggcgtttgtgctgcccgaccctttctcctgtccggatgcgcagggcagggcctgagccgtcgagctgcacccacagcaggctgcctttggtgactcaccgggtgaacgggggcattgcgaggcatcccctccctgggtttggctcctgcccacggggctgacagtagaaatcacaggctgtgagacagctggagcccagctctgcttgaacctattttaggtctctgatccccgcttcctctttagactcccctagagctcagccagtgctcaacctgaggctgggggtctctgaggaagagtgagttggagctgaggggtctggggctgtcccctgagagaggggccagaggcagtgtcaagagccgggcagtctgattgtggctcaccctccatcactcccaggggcccctggcccagcagccgcagctcccaaccacaatatcctttggggtttggcctacggagctggggcggatgacccccaaatagccctggcagattccccctagacccgcccgcaccatggtcaggcatgcccctcctcatcgctgggcacagcccagagggtataaacagtgctggaggctggcggggcaggccagctgagtcctgagcagcagcccagcgcagccaccgagacacccaagtttgtacaaaaaagcaggctgccaccatgtacagcgctccaagcgcctgcacatgcctgtgtctgcacttcctgctgctgtgcttccaggtgcaggtgctggtggccgaggagaacgtggattttaggatccacgtggaaaaccagaccagagccagagatgatgtgagcaggaagcagctgcggctgtaccagctgtattcaaggaccagcggcaaacacatccaggtgctgggacggaggatctccgccagaggggaggacggtgacaagtacgcccagctgcttgtggagactgatacatttggctcccaggtgagaatcaagggcaaggagaccgagttttacctgtgcatgaacaggaaaggcaagctggtgggcaagccagacgggaccagcaaggagtgcgtgttcatcgagaaggtgctggagaacaactataccgccctgatgtccgccaagtactctggctggtacgtgggattcacaaagaagggaaggcctcgcaagggccctaagacccgggagaaccagcaggatgtccacttcatgaagagataccctaagggacagcccgaactgcagaagcccttcaagtacacaactgtgacaaagagaagcaggagaatcagaccaactcaccccgcctgaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaactagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 5 - DNA, 5214 bp, synthetic construct
  source 1..5214: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatgtattcagcgccctccgcctgcacttgcctgtgtttacacttcctgctgctgtgcttccaggtacaggtgctggcggccgaagagaacgtggacttccgcatccacgtggagaaccagacgcgggctcgggacgatgtgagccgtaagcagctgcggctgtaccagctctacagccggaccagtgggaagcacatccaggtcctgggccgcaggatcagtgcccgcggcgaggacggggacaagtatgcccagctcctagtggagacagacaccttcggtagtcaagtccggatcaagggcaaggagacggaattctacctatgtatgaaccggaaaggcaagctcgtggggaagcccgatggcaccagcaaggagtgtgtattcattgagaaagtcctggagaacaactatacagccctgatgtccgccaagtactctggctggtacgtgggcttcaccaagaaggggcggccccggaagggtcccaagacccgggagaaccagcaggacgtgcacttcatgaagcgctaccccaagggacaggcagagctgcagaagcccttcaagtacaccacggtgaccaagaggtcccggcggatccgccccacgcaccccggctagacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 6166 bp, synthetic construct
  source 1..6166: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtattcagcgccctccgcctgcacttgcctgtgtttacacttcctgctgctgtgcttccaggtacaggtgctggcggccgaggagaacgtggacttccgcatccacgtggagaaccagacgcgggctcgggacgatgtgagccgtaagcagctgcggctgtaccagctctacagccggaccagcgggaagcacatccaggtcctgggccgcaggatcagtgcccgcggcgaggacggggacaagtatgcccagctcctagtggagacagacaccttcggtagtcaagtccggatcaagggcaaggagacggaattctacctgtgtatgaaccggaaaggcaagctcgtggggaagcccgatggcaccagcaaggagtgtgtgttcattgagaaggtcctggagaacaactacacagccctgatgtccgccaaatactctggctggtacgtgggcttcaccaagaaggggcggccacggaagggtcccaagacccgggagaaccagcaggacgtgcacttcatgaagcgctaccccaagggacaggcagagctgcagaagcccttcaagtacaccacggtgaccaagaggtcccggcggatccgccccacgcacccaggctagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - protein, 173 aa, synthetic construct
AEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSR